(19) United States
(12) Patent Application Publication
(10) Pub. No.: US 2006/0079923 A1
(43) Pub. Date: Apr. 13, 2006

(54) Aneurysm treatment using semi-compliant balloon

(76) Inventors: Manik Chhabra, Stanford, CA (US); Neema Hekmat, Mountain View, CA (US); Peter Johnson, Mountain View, CA (US); Amy Lee, Riverside, CA (US); Lipkong Yap, Stanford, CA (US)

Correspondence Address: BELL & ASSOCIATES, 416 FUNSTON ST., SUITE 100, SAN FRANCISCO, CA, 94118, US

(21) Appl. No.: 11/200,947

(22) Filed: Aug. 9, 2005

Related U.S. Application Data
(60) Provisional application No. 60/600,074, filed on Aug. 9, 2004.

(51) Int. Cl. A61M 29/00

(52) U.S. Cl. 606/192

(57) ABSTRACT

A device for occluding an aneurysm comprising: a detachable, semi-compliant, radially-expanding balloon mounted on a catheter, wherein the balloon is in fluid communication with the catheter, wherein the balloon comprises a plurality of micropores, and wherein the micropores in the balloon allow expression of a bio-adhesive fluid at a defined pressure from the inside to the outside of the balloon.

The present application claims priority to U.S. Provisional Patent Application Ser. No. 60/600,074 entitled “Aneurysm Treatment Using Semi-Compliant Balloon”, filed Aug. 9, 2004, which is herein incorporated by reference in its entirety for all purposes.

### BACKGROUND

Intracranial Aneurysms

An aneurysm is an out-pouching or dilatation of a blood vessel within the body. It is generally believed that the aneurysm develops from an initial small lesion in the vessel wall. While there are many different stimuli proposed for this lesion, such as mechanical tearing due to highly concentrated wall stress or immune dysfunction, the propagation of the aneurysm from a small tear to a large dilatation is generally understood.

Physiology of Aneurysms

Arterial walls are constructed from three distinct layers. The innermost layer, adjacent to the lumen where blood flows, is called the intima. It is composed mostly of flat endothelial cells that regulate the majority of the functions of the vessel wall by sensing stimuli on the lumen. Next to these cells lies a thin basilar membrane. The second layer is called the media, which is composed of smooth muscle cells oriented circumferentially around the artery and of matrix proteins (elastin and collagen) produced by the smooth muscle cells. Elastin and collagen differ highly in their material properties and in their roles in providing strength and shape to the vessel. Elastin is highly compliant but exhibits a lower yield strength, while collagen is much more stiff but stronger in tension. Elastin is oriented in sheets called lamellar units. These sheets are wrapped tightly around the lumen and absorb the majority of the stress or arterial pressure waves. Collagen fibers are woven into the matrix, but they are generally in more of a kinked configuration during normal pressures; they are straightened out during expansion, but it is not common for a vessel to expand to the point that it is stretching and stressing collagen fibers in a straight configuration. It is this second layer, the media that is most affected and directly involved in the formation of an aneurysm. The third and outermost layer of the artery is called the adventitia. It is made up of mostly collagen fibers and is also connected to the tissues surrounding the artery, helping to hold the vessel in place as it pulsates through the cardiac cycle.

When a lesion forms on an arterial wall, the immediate physiological reaction is to heal it as quickly as possible. Aneurysm propagation has been described as “slow rupture.” For reasons not clearly understood, elastin and smooth muscle cells basically disappear from the media and the collagen that acted only as a sort of safety jacket becomes the stress-bearing element of the wall. Small hemorrhages are constantly repaired by adding collagen fibers. In normal pathologies, collagen has a very high tensile strength due to cross-links that form between fibers. These cross links form as the collagen fibers mature over a period of 300 days. During this maturation, the collagen fibers are easily ordered and aligned to give a high tensile strength because they are typically not bearing much of the load. In the case of an aneurysm where there is a lack of smooth muscle cells and elastin to bear pressure loads, collagen fibers are never allowed to reorder and mature. Thus small ruptures continue to form and be repaired without any effective restructuring, and an aneurysm forms out from the normal artery path. Aneurysms are described as having a fundus, or dome, and a neck. The wall thickness varies from thick to thin from the neck to the fundus. Measurements have shown the thickness of the fundus wall to be an average of 2.4% of the radius of the aneurysm. There is also a lack of endothelial cells lining the wall at the fundus. One study has reported finding them in only 10% of the fundi of examined aneurysms.

Prevalence, Location, and Symptoms

Aneurysms that appear in the vasculature of the brain are known as intracranial aneurysms. There are two main types of aneurysms that form in the brain: saccular, or berry, and fusiform. Saccular aneurysms comprise 90% of intracranial aneurysms; they are round sacs that protrude off of one side of an artery, while fusiform aneurysms are generally more amorphous and extend circumferentially from the path of the artery, more closely resembling the giant aneurysms that form along the abdominal aorta. 90% of intracranial aneurysms occur at bifurcations on or near the Circle of Willis, an interconnected circular blood vessel found at the base of the brain. Most aneurysms are the result of abnormal thinning of the artery wall and subsequent loss of the important structural fiber elastin. Intracranial aneurysm prevalence has been linked to heredity, aging, smoking, and excessive alcohol use.

Most intracranial aneurysms are asymptomatic until rupture. Occasionally they manifest themselves through dizziness or headaches but most go undetected unless diagnosed as a result of a non-specific screen, such as magnetic resonance angiography after head trauma. Rupture of an aneurysm results in bleeding into the space between the brain and the arachnoid membrane that surrounds it. This is known as subarachnoid hemorrhage (SAH). In the United States, ten to fifteen million people are estimated to have saccular intracranial aneurysms, and each year approximately 30,000 saccular aneurysms rupture Among victims, there is a mortality rate of about 50% within the first month; 10-15% die before even reaching the hospital. About half of those who survive the first month experience permanent neurological defects and disabilities.

In SAH, bleeding occurs from the ruptured artery into the cerebral spinal fluid for a few seconds until the pressure in the spinal fluid becomes greater than that of the artery and stops blood outflow or collapses the vessel. Causes of death in SAH include ischemia of the brain tissue fed by the vessel on which the rupture occurs as blood flow is significantly reduced by regulatory mechanisms within the body. SAH also causes a rapid increase in intracranial pressure, which in turn may cause global ischemia, brain hemorrhage, or other disruption of more fragile structures in the brain stem.

Medical Treatment of Intracranial Aneurysms

Approximately 50% of previously ruptured and healed aneurysms rebleed with 6 months. These rebleeds are fatal in 70-90% of cases. A rupture should be treated within 24-48 hours to effectively prevent rebleeding. Treatment is also indicated for detected unruptured aneurysms that fit certain criteria such relative young age of patient, a diameter of 5 mm or higher, and family history of ruptured aneurysms. Lifestyle of the patient also comes into play. Cigarette smoking and excess alcohol consumption are known to increase the chance of rupture. The decision to treat unruptured aneurysms is ultimately one made by balancing the percentage risks of rupture with the percentage risks of surgical complications; if an aneurysm, based on risk factors discussed above, has a 5% chance of rupturing and there is a 7% chance of surgical complications, no treatment will be attempted.

Intracranial aneurysms have traditionally been treated by surgical clipping during a craniotomy. In this procedure, the neurosurgeon approaches the aneurysm through a hole in the skull and places a metal clip over the neck, effectively sealing off the at-risk rupture site from blood flow. Clipping is considered an effective method—over 90% of the aneurysms treated with this approach are obliterated after surgery. Nine years ago, endovascular coiling became an alternative to the clipping approach with the FDA approval of Guglielmi detachable coils (GDC; Target Therapeutics, Fremont Calif.). In this procedure, a neuroradiologist inserts a catheter into the femoral artery (the brachial artery is the more common entry point in Europe) and weaves it up to the aneurysm site in the brain.

Microcatheters that are applicable to these locations in the brain generally must have a profile of no more than 1 mm. A series of platinum coils are expelled into the saccule from the catheter until a tight ball is formed. A thrombus then forms around the coils by physiologic mechanisms and the aneurysm is obliterated. It has even been observed in some cases that a thin layer of endothelium actually grows across the opening of the aneurysm after thrombogenesis has occurred. A recently completed trial has shown a 22.9% relative risk reduction for death and dependency after one year using coiling over clipping techniques. Economically, coiling makes sense as well. A study on the treatment of unruptured aneurysms found that coiling resulted in an average five-day reduction of length of stay and $13,000 per patient in cost savings over clipping. In 1999, 15% of all intracranial aneurysm surgeries in the U.S. were coiling procedures, with a 7% annual growth rate predicted since then. Numbers in Europe are considerably higher because the procedure was introduced earlier and so has already found higher acceptance.

Wide Neck Aneurysms

One major limitation of endovascular coiling is that it is insufficient in treating wide neck intracranial aneurysms. A wide neck aneurysm is defined as one having a neck that is greater than 4 mm in diameter or a neck diameter that is greater than half the size of the maximum diameter of the aneurysm. The problem is that as coils are expelled into the aneurysm they can be washed out by the higher flows that are present with a wider neck. There have been variations in the coiling regiment designed to hold the coils in until they can be packed tightly enough to prevent slip out; these will be discussed later in the report. Despite new innovations, clipping is still the current method of choice for treating wide neck aneurysms. However, the data on the efficacy of endovascular treatment in lowering risk and reducing cost strongly suggests that if a satisfactory method of treating wide neck aneurysms endovascularly can be developed it would find acceptance similar to that of coiling for narrow necks.

Approximately 30% of all saccular intracranial aneurysms are classified as wide neck, translating to about 9,000 potential cases per year.

Clinical Problems

Rupture of intracranial aneurysms occurs almost uniformly at the apex of the fundus due to failure of the collagen wall. The average chronic tensile strength of this wall has been measured in various experimental procedures to be around 0.25 MPa. Assuming static flow and spherical geometry and using the simple spherical hoop stress formula shown below that correlates wall stress σ with hydrostatic pressure P, radius R, and wall thickness t, it has been determined that mean in vivo stress is sufficient to rupture the wall as it weakens through stress relaxation cycles that correlate with the pulsatility of blood flow. (See Equation i)

$$σ = \frac{1}{2} \frac{PR}{t} \qquad ( i )$$

Hydrostatic pressure required to induce a wall stress of the above magnitude would be about 90 mm Hg, which is physiologically seen. This suggests that collagen walls are at or near the breaking point constantly and reinforces the idea that the walls are constantly tearing and repairing themselves. At some point, the tear grows too large for self repair, and rupture occurs in direct result of fluid pressure-induced wall stress.

Obviously any interventional therapy for these aneurysms must address this problem. Treatment could consist of increasing the tensile strength of the wall, possibly by simply increasing thickness, t, or more commonly, decreasing the stress sigma on the wall by lowering local pressure or shear forces. Successful therapy would prevent rupture and further propagation by accomplishing one or both of these objectives with minimal risk.

The most common means of treating these aneurysms is to fill the space with either a temporary or permanent material. An example of this is the coiling method described earlier. This process depends on the development of a natural thrombus as well to strengthen the occlusion. It is also possible to greatly diminish wall stress by merely altering flows into the aneurysm fundus. Imbesi et al. showed that the mere placing of a stent in the lumen of the artery from which an aneurysm arose significantly decreased the stress felt by the wall of the aneurysm (Imbesi et al. (2003) Am. J. Neuroradiol. 24: 2044-2049).

While there are many technologies and patents specifically geared to address the needs of this market, there is a significant opportunity to develop a novel device that will exhibit long-term permanent occlusion and elicit a desirable biological response while decreasing risk and complexity of the procedure. There is currently not a widely accepted effective endovascular device for occluding wide neck aneurysms.

Market

Currently, around 25,000 procedures are done per year in the United States to obliterate intracranial aneurysms. Of these, it is estimated that about 7,500 are performed on wide neck aneurysms. Traditional coiling procedures carry an estimated cost of $16,000. If this amount is used to estimate the cost of a new endovascular therapy for wide neck aneurysms, the potential market for the treatment of wide necks can be estimated roughly as $120 million per year. The specific market for treating wide necks endovascularly over clipping procedures is a segment of this, and its size over time would depend on the success of the endovascular treatment over traditional surgical clipping. If a new treatment could be designed that provided a treatment not only for wide neck aneurysms, but also for narrow neck aneurysms, it would reach a much larger market size. The market potential for such a device would be 2-3 times that of the wide neck aneurysm market alone. Additionally, as diagnostic capabilities improve these previous statistics may become irrelevant as it will become more common to treat unruptured aneurysms when there is less surgical risk involved. Such treatments could potentially be applied to a significant portion of the 10 million Americans believed to have at least one intracranial aneurysm.

### BRIEF DESCRIPTION OF THE INVENTION

The invention is a device for treating an aneurysm. In one embodiment the device is used for occluding an aneurysm, the device comprising: a detachable, semi-compliant, radially-expanding balloon mounted on a catheter, wherein the catheter comprises a catheter body defining at least one interior lumen, wherein the balloon is in fluid communication with at least one lumen defined within the catheter, wherein the balloon comprises a plurality of micropores, wherein the micropores in the balloon allow expression of an adhesive fluid at a defined pressure from the inside to the outside of the balloon. In one preferred embodiment the balloon is non-compliant.

In another preferred embodiment the micropores in the balloon are disposed unevenly upon the surface of the balloon. In a more preferred embodiment, the majority of the micropores are disposed on the upper hemisphere of the balloon. In another embodiment, the micropores are disposed over an area of not more than 50% or the surface of the balloon. In yet another embodiment, the micropores are disposed over an area of not more than 30% or the surface of the balloon. In a still further embodiment, the micropores are disposed over an area of not more than 10% or the surface of the balloon.

In another preferred embodiment the total combined surface area of the micropores is not more than 0.5% of the total surface area of the balloon. In another embodiment the total combined surface area of the micropores is not more than 1% of the total surface area of the balloon. In a still further embodiment, the total combined surface area of the micropores is not more than 2% of the total surface area of the balloon. In a still further embodiment, the total combined surface area of the micropores is not more than 5% of the total surface area of the balloon.

The invention further provides a device for occluding an aneurysm wherein the device comprises a fluid. In a preferred embodiment, the fluid is a bio-adhesive fluid that solidifies under physiological conditions. In a more preferred embodiment, the fluid is a polymerizing material. In a most preferred embodiment, the fluid is a cyanoacrylate material. In one embodiment, expression of the bio-adhesive fluid from the micropores requires a minimum interior pressure of 30 mm Hg. In another embodiment, expression of the bio-adhesive fluid from the micropores requires a minimum interior pressure of to 60 mm Hg. In a still further alternative embodiment, expression of the bio-adhesive fluid from the micropores requires a minimum interior pressure of to 80 mm Hg. In a yet further embodiment, expression of the bio-adhesive fluid from the micropores requires a minimum interior pressure of to 100 mm Hg. In another embodiment, expression of the bio-adhesive fluid from the micropores requires a minimum interior pressure of to 120 mm Hg. In a still further alternative embodiment, expression of the bio-adhesive fluid from the micropores requires a minimum interior pressure of to 160 mm Hg.

Another embodiment of the invention provides a device for occluding an aneurysm wherein the shape of the balloon is approximately torroidal. Another embodiment provides a device wherein the shape of the balloon is disc-shaped wherein the diameter of the disc is greater than the thickness of the disc. A more preferred embodiment provides the disc-shaped balloon possessing a concave lower surface.

A further embodiment of the invention provides a device for occluding an aneurysm comprising a catheter wherein the catheter comprises a major lumen and a minor lumen wherein the major lumen is adapted for delivery of the bio-adhesive fluid and wherein the minor lumen is adapted for containment of an electrically conductive wire. In one preferred embodiment the device for occluding an aneurysm further comprises, at or near the attachment point of the balloon and the catheter body, a steel coupling detachably joining the balloon and the catheter body.

The invention further provides a device for occluding an aneurysm comprising a balloon having micropores wherein the diameter of the micropores is between 1 μm and 10 μm.

The invention further contemplates a method of using a device for occluding an aneurysm in an individual, the method comprising the steps of: i) providing an individual at risk for having an aneurysm; ii) providing a device, the device comprising a double lumen catheter, the catheter comprising a first tube, a second tube, a steel couple, a non-steel couple, and an electrically conducting wire, the first tube having at least one side hole and a lumen and the second tube having at least one side hole and a lumen, a semi-compliant balloon, the balloon having a plurality of micropores disposed upon the surface of the balloon; iii) inserting a guidewire through a blood vessel of the individual into the aneurysmal space; iv) using the guidewire as a rail inserting the device through the blood vessel; v) advancing the device until the balloon is positioned in the aneurismal space; vi) injecting a radio-opaque composition into at least one lumen of the device; vii) visualizing the radio-opaque composition in the aneurysmal space; viii) withdrawing the radio-opaque composition from the device; ix) injecting a adhesive fluid into the device at a pressure suitable for inflating the balloon and fixing the balloon against the interior wall of the aneurysm; x) placing an electrode on the individual, the electrode being in electrical communication with the ground attachment of a voltage source; xi) applying a potential difference to the electrically conducting wire thereby causing electrolysis of the steel couple and releasing the steel couple from the non-steel couple; thereby treating the aneurysm.

### BRIEF DESCRIPTION OF THE DRAWINGS

FIG. 1 illustrates an embodiment of the invention and shows an electrolytically detaching balloon and detaching elements.

FIG. 2 illustrates features of the prior art GDC detaching system.

FIG. 3 illustrates an exemplary embodiment of the balloon of the invention.

FIG. 4 illustrates an experimental setup used to validate a prototype of a replica aneurysm at a scale of five-times greater than the expected size of the invention.

FIG. 5 illustrates a detail of the experiment that modeled occluding a replica aneurysm.

FIG. 6 illustrates an experimental setup used to validate a prototype of a replica aneurysm at a scale of two-times greater than the expected size of the invention incorporating an electrolytic detaching system.

FIG. 7 illustrates an experimental prototype of the invention to test pressure thresholds.

FIG. 8 illustrates a plot of the test data and linear trends of three separate experiments.

FIG. 9 illustrates a plot of targeted correlation between viscosity and resulting pressure.

FIG. 10 illustrates a photomicrograph of pores created using a sewing needle.

FIG. 11 illustrates a photomicrograph of pores created using a hypodermic needle.

FIG. 12 illustrates a photomicrograph of pores created using a fine wire having a diameter of about 30 μm.

FIG. 13 illustrates how a prototype was assembled.

FIG. 14 illustrates different embodiments of the invention.

FIG. 15 is a diagram of the reaction of RGD ligand and its immobilization on polymer surfaces (adapted from Kessler et al, 2003 Biomaterials 2003; 24, 4385-4415).

### DETAILED DESCRIPTION OF THE INVENTION

The invention encompasses a device for occluding aneurisms, specifically wide neck cerebral aneurysms. The invention further encompassed methods for using the device of the invention for treating aneurisms such as wide neck and the more common narrow neck saccular aneurysms.

The structure of the device generally includes a detachable, semi-compliant, radially-expanding microporous balloon mounted on a catheter. Micropores in the balloon material allow for the controlled flow of fluid at certain pressure levels. The balloon comprises a semi-compliant material that is able to easily deform in a desired direction. Deforming the balloon in a desired direction can better let an operator controllably expand the balloon in an aneurysm in cases where the aneurysm has walls with differential thickness and is vulnerable to symmetrical forces within. In certain embodiments, the surface of a balloon may be required to expand evenly. In other embodiments, the balloon comprises a material that is non-compliant and deforms slightly when expanded by an operator. A non-compliant balloon is desirable as the outer wall of the balloon is less likely to form adhesions when in contact with the inner wall of the aneurysm. A non-compliant balloon is also desirable as the balloon is much less likely to rupture in a region of localized thinning of the balloon wall upon expansion. In addition the micropores conserve their size and aperture area if the balloon comprises a non-compliant material.

In one aspect, the balloon has a diameter of not more that 10 mm or a surface area of not more than 315 mm² or a volume of not more than 525 mm³. Preferably, the diameter can be between 0.5 mm and 10 mm, such as 0.5 mm, 1 mm, 1.5 mm, 2 mm, 2.5 mm, 3 mm, 4 mm, 5 mm, 6 mm, 7 mm, 8 mm, 9 mm, 10 mm, or any diameter within that range; or the surface area can be between 3.14 mm² and 315 mm², such as 0.78 mm², 3.14 mm², 7.07 mm, 12.57 mm², 19.63 mm², 28.27 mm, 50.26 mm², 78.54 mm², 113.1 mm, 153.9 mm², 201.6 mm², 254.5 mm², 315 mm² or any area within that range; or the volume can be between 0.065 mm³ and 525 mm³, such as 0.065 mm³, 0.524 mm³, 1.77 mm³, 4.19 mm³, 8.18 mm³, 14.14 mm³, 33.51 mm³, 65.45 mm³, 113.1 mm³, 179.58 mm³, 268.07 mm³, 381.69 mm³, 525 mm³, or any volume within that range.

In certain important embodiments, the micropores are distributed unevenly upon the surface of the balloon, for example the micropores may be distributed only on the upper portion of the balloon (the portion directly opposite the catheter entrance point). The pores may be disposed over the entire upper hemisphere of the balloon (50% coverage) or a smaller area, such as 40%, 30%, 20%, 10%, 5% or less coverage. Pores may be distributed evenly or randomly over a particular area or in any desirable pattern such as in concentric circles around the “pole” of the balloon.

In certain embodiments, the balloon may be inflated with a bio-adhesive fluid, for example, a biocompatible polymer such as a polymerizing cyanoacrylate material, that will be expressed, under appropriate pressure, through the pores and will secure the balloon to the interior of the aneurysm site and also harden within the balloon, causing permanent occlusion. In certain specific embodiments, a hardening substance such as cyanoacrylate may be supplied to the balloon through the larger lumen of a double-lumen catheter. Detachment of the catheter from the balloon may be achieved via a coupling that joins the distal end of the catheter with the balloon. The coupling may be made of, for example, stainless steel, or nickel-titanium alloy. The proximal end of the device is handled by the physician. A wire (for example, a copper wire) may be housed within the smaller lumen of the catheter and insulated by the catheter until it is soldered to the steel couple. In an alternative additional embodiment, the distal end of the catheter can extend through the lumen of the balloon, as illustrated in FIG. 13, and the distal open end can additionally be sealed to prevent leakage of bio-adhesive fluid from within the catheter lumen. For example, a silicone rubber cap having a self-sealing distal aperture is used to seal the distal open end but that allows the catheter to be threaded on a thin guidewire via the distal aperture.

In use, the device may be deployed to occlude an aneurism using methods that generally include the following steps or variations thereupon. Steps common to catheter intervention procedures, such as entry into an artery or vein, visualization of target vasculature by radiopaque bolus injection have been omitted to emphasize steps important to this procedure.

An electrode is placed on the patient and connected to the ground attachment of a voltage source. A guidewire is advanced through the cerebral vasculature and into the aneurysmal space. The balloon catheter is threaded over the end of the guidewire and advanced along it into the aneurysm. The catheter can be threaded over the guidewire, the guidewire being positioned in the lumen of the catheter. In the alternative, the catheter can comprise a guidewire mount, for example, a series of loops or a tubular structure, upon the catheter exterior surface and the catheter is guided through the blood vessels the guidewire being placed through the loops or the tubular structure. This alternative has the advantage in that the guidewire is not placed in the lumen of the catheter and cannot compromise the integrity of the system when the sealing fluid is placed under a positive pressure for extrusion or expression from the catheter and the balloon. Contrast solution is injected into the balloon to confirm that the size of the balloon is appropriate and that it can be properly positioned in the neck of the aneurysm. Once proper sizing has been confirmed, contrast solution is pulled back out of the balloon. A calculated volume of radiopaque/cyanoacrylate formulation, the sealing adhesive fluid material, is injected into the catheter and is chased with saline up into the balloon using a syringe equipped with a pressure gauge. The balloon is positioned against the aneurysm wall and cyanoacrylate is forced out of the pores, fixing the balloon to the interior wall of the aneurysm.

After curing, which generally takes about 5-10 minutes, a current is conducted through the copper wire and into the steel couple, causing electrolysis to occur.

Complete electrolytic detachment will be indicated by a sudden drop in current and by visual confirmation through angiography. Upon detachment, the proximal end of the catheter is withdrawn.

Balloon Shape

An important consideration is to prevent the balloon from inflating and occluding the parent vessel from which the aneurysm emerges. This becomes even more of an issue if a detached section of catheter is also hanging from the portion of the balloon that is proximal to the operator. Any structure protruding into the main flow of the parent artery may increase the occurrence of a thrombus or other occlusions. For this reason, various embodiments include a balloon with a flat or concave proximal portion.

The shape of the top side of the balloon is also carefully designed. It is believed that the majority of aneurysms rupture near the apex, and in view of the fact that samples of artery wall taken from the apex have been shown to be considerably weaker than those taken from near the neck of the aneurysm. For this reason, any stress placed on the aneurysm wall should be focused on the neck region rather than the apex. Thus various designs avoid a balloon that expands upwards into the apex.

One preferred embodiment includes a balloon that expands radially outward rather than upwards into the apex. The balloon may be place near the neck of the aneurysm prior to inflation. By taking a balloon roughly spherical in shape and constraining the ends of it along the catheter, a donut-like (torroidal) inflatable balloon may be formed. This shape allows for minimal intrusion of the detached section of the catheter into the lumen of parent arteries. It also places stress on the neck of the aneurysm because expansion will occur radially.

Balloon Material

In choosing a material for the balloon, several factors should be considered, including manufacturability, expansion predictability, risk of rupture, ability to seal off the neck of the aneurysm, and ability to allow controlled delivery of adhesive to the aneurismal space.

In certain embodiments, a soft, elastic, compliant balloon may be used. However, a compliant balloon may not function as well as a non-compliant or semi-compliant balloon for delivering polymer and would have less predictable spatial expansion rates. One potential problem in using a compliant balloon would be that it would not be able to inflate without also increasing the size of the pores, preventing a physician from being able to deliver the polymer to the walls of the aneurysm at a predictable rate and also possibly resulting in stray emboli that can migrate or be conducted to another organ. On the other hand, a non-compliant balloon would have more predictable expansion and pores would almost entirely retain their original unexpanded size that adhesive could be delivered at a threshold pressure above that required to inflate the balloon out to the walls of the aneurysm. This was demonstrated by Applicants of the disclosed invention by testing several non-compliant microporous balloons obtained from Advanced Polymers Inc, a medical balloon manufacturing company located in Salem, N.H. It was found that the balloons could be fully inflated and then fluids of different viscosities could be sequentially forced out of the balloons by applying higher pressure.

In certain embodiments, in order to decrease the risk of physician-induced rupture, the balloon may be oversized for the space they will be inserted into, and inflated only until they become flush with the artery walls and/or the wall of the aneurysm. Thus they would fill the space well enough to allow polymer to seep out onto the aneurysm walls, locking them in place while they harden, while decreasing the likelihood of rupture.

Both types of material have advantages in different ways. Semi-compliant balloons exhibit some of the properties of both compliant and non-compliant balloons. Semi-compliant balloons are softer than compliant balloons, and can expand more to fill the space in which they are placed, but they still retain enough rigidity that pores would not expand uncontrollably. The material could be constrained to inflate into a pre-defined shape, for example, an approximately torroidal shape. For example, a balloon can be made from a semi-compliant, low durometer urethane material or equivalent thereof, such as stereolithography (SLA) resin, silicone rubber, latex, or the like; a biological material or compound, such as collagen, keratin, fibrin, cellulose, or the like, and combinations thereof.

Note that although certain balloon materials are used in this disclosure as examples, the disclosure is not intended to limit the invention to any particular material, and any material known in the art may be used with the present invention.

Balloon Porosity

Microporous balloons have been used in the medical device industry for a variety of reasons. Most often they are used as a method of controlled drug delivery to artery tissue. A preferred microporous balloon comprises a formulated pore size and pore density and that allow it to be inflated to its maximum size before fluid is expelled through the pores. The pressure limit to which the balloon can be inflated without expelling fluid will be referred to hereafter as threshold pressure. Because stray emboli are a problem with current polymer embolism procedures, it was felt to be imperative to be able to expand the balloon to the aneurysm wall without prematurely forcing any cyanoacrylate out of the pores. The location of the pores on the balloon surface is also an important issue to prevent stray emboli from being released into the circulatory system. Pores can been created only on the upper hemisphere of the balloons in an effort to keep the adhesive polymer above and along the sides of the balloon.

The pores can be created using micro-excision devices such as, but not limited to, devices that use laser technology, devices that use ultrasound to create pores or apertures, devices that use radio or wireless technology, devices that use microbial organisms that are modified to secrete enzymes that can create pores, or the like.

While the pores used in the model as disclosed in the Examples were made by hand, such pores can be manufactured and created commercially when the device is produced on a larger scale for animal testing and in human subjects. Microporous balloons of known pore size, pore density, and overall surface area can be purchased from, for example, Advanced Polymers Inc. (Salem N.H.) and can be used to create some baseline equations and algorithms for determining target pore sizes and pore densities for commercial manufacturing. Details of these tests are disclosed in the Examples section along with baseline equations.

The total combined surface area of the pores relative to the surface area of the balloon can be at least 0.5% of surface area of the balloon. For example, for a balloon with a surface area of 78.54 mm², 0.5% (the total combined surface area of the pores) is 0.3927 mm²; for a balloon with a surface area of 100 mm², 0.5% (the total combined surface area of the pores) is 0.5 mm².

In a preferred embodiment the combined surface area of the pores relative to the surface area of the balloon is at least 1.0% of the surface area of the balloon. For example, for a balloon with a surface area of 78.54 mm², 1.0% (the total combined surface area of the pores) is 0.7854 mm²; for a balloon with a surface area of 100 mm², 1.0% (the total combined surface area of the pores) is 1.0 mm².

In the alternative, the combined surface area of the pores relative to the surface area of the balloon is at least 2.0% of the surface area of the balloon. For example, for a balloon with a surface area of 78.54 mm², 2.0% (the total combined surface area of the pores) is 1.57 mm²; for a balloon with a surface area of 100 mm², 2.0% (the total combined surface area of the pores) is 2.0 mm².

In the alternative, the combined surface area of the pores relative to the surface area of the balloon is at least 5.0% of the surface area of the balloon. For example, for a balloon with a surface area of 78.54 mm², 5.0% (the total combined surface area of the pores) is 3.927 mm²; for a balloon with a surface area of 100 mm², 5.0% (the total combined surface area of the pores) is 5.0 mm².

In one aspect a 2% total combined surface area of the pores relative to the surface area of the balloon (“open area”) can establish a threshold pressure of about 120 mm Hg for a fluid of 3 centipoise (cP), the viscosity for the glue formulation that were used in the tests described below. Glues with other viscosities, such as with lower (<3 cP) or with higher viscosities (>3 cP) are known to those of skill in the art and the percentage open area of the balloon surface can be determined empirically.

A balloon having pores with diameters of about 10 μm can be made by hand using thin steel wire. These are approximately 10 times the size of the pores that can be cut into the balloons using a commercial cutting device, such as those disclosed above. This went into consideration while performing our tests, and as a result we made fewer pores than we intend to on the final prototype. The pores may be disposed over the entire surface of the balloon (about 100% coverage) or a smaller area, such as about 90%, about 80%, about 75%, about 70%, about 66%, about 60%, about 50%, about 40%, about 33%, about 30%, about 25%, about 20%, about 10%, about 5%, about 3%, about 2%, about 1% or less coverage.

An important feature of the present invention is that the pores of the balloon, in certain embodiments, are not distributed evenly about the surface area of the balloon, but are localized to certain regions of the surface of the balloon. For example, the micropores may be distributed only on the upper portion of the balloon (the portion directly opposite the catheter entrance point). The pores may be disposed over the entire upper hemisphere of the balloon (about 50% coverage) or a smaller area, such as about 40%, about 30%, about 20%, about 10%, about 5%, about 3%, about 2%, about 1% or less coverage. In another alternative example, the micropores may be disposed only on a portion of the balloon that is on the side of the balloon, relatively perpendicular to the catheter entrance point. Pores may be distributed evenly or randomly over a particular area or in any desirable pattern such as in concentric circles around the “pole” of the balloon. Such local distribution has important benefits in that it reduces the probability that the acrylic glue (or the like) will leak from the interior of the aneurism into the blood vessel, where it could cause thrombosis. Another advantage is that local extrusion of the adhesive at the “upper” surface of the embolism allows bonding and attachment to initiate at the apex of the embolism, which is considered to be the weakest point of many emboli. As adhesive continues to be extruded from the balloon, the outer surface of the balloon adheres to the inner surface of the embolism over in increasing area until the adhesive begins to harden and set, occluding the embolism. Local distribution of the micropores therefore reduces the dangers of leaking adhesive that may cause thrombosis, and produces a balloon with superior adhesive qualities.

Detachment of the Balloon

Existing patents and disclosures describe simple mechanical detachment methods where a user can pull the proximal end of a delivery catheter and remove it from the distal balloon. For ease of manufacturing and simplicity of use, a simple mechanical couple detachment device can be used to detach the delivery catheter from the balloon.

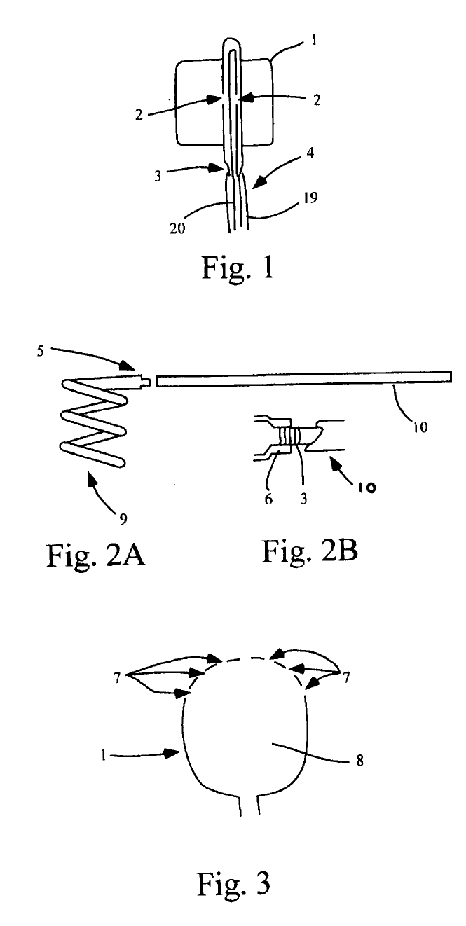

The present invention provides a detaching device that comprises controlled detachment and uses an electrolysis reaction and a hollow stainless steel tube as a dissolvable junction (see FIG. 1). FIG. 1 illustrates a balloon (1), side holes (2) in the catheter walls for extruding and expressing a sealing adhesive fluid material into the lumen of the balloon, a stainless steel couple element (3), and a double lumen catheter (4), the double lumen catheter comprising a first tube and a second tube, the second tube being disposed longitudinally within the lumen of the first tube. Steel electrolysis is used in most Guglielmi detachable coil (GDC) coiling procedures as a method of detachment and is an FDA-approved detachment mechanism (see FIG. 2; redrawn from Target Therapeutics, Fremont Calif.). FIG. 2 illustrates features of the GDC detaching system (5), a steel male couple element (3), and a female couple element comprising non-steel material (6).

The GDC system consists of a soft platinum coil soldered to a stainless steel delivery wire. When the coil is positioned a current is applied to the delivery wire. The current dissolves the stainless steel delivery wire proximal to the platinum coil by means of electrolysis. In conventional use platinum coils are soldered onto a steel wire that pushes them through a microcatheter. The steel is exposed to the bloodstream right above the microcatheter before the soldering. An electrode is placed on the patient setting their blood voltage at ground level, while about four to five volts and about 90 mA of current are applied to the steel wire proximal to the percutaneous entry point of the catheter. The ferrous ions in the steel are slowly drawn out of the metal structure by the blood ions and the junction dissolves, releasing the coil.

In the present invention, a similar scientific concept is applied to a small stainless steel hypotube. A wire threaded through the small lumen of the catheter and is attached to the couple. Both sides of the delivery catheter are fit and glued onto the steel coupling, and a current is applied to complete the detachment process and the distal balloon portion remains in the aneurysm while the proximal portion can be removed from the vasculature. Using 0.04″ diameter steel tubes electrolysis and detachment can be performed within two to three minutes.

The wire that conducts the electrical current can be any electrically conductive metal or suitable polymeric compound. The wire can comprise any electrically conducting metal, such as steel, copper, platinum, silver, gold, palladium, or the like. Alternatively, the wire can comprise an electrically conductive plastic or polymer composition, such as polyolefin or polyethylene polymer and an electrically conductive carbon black as described in U.S. Pat. No. 4,562,113 or polyurethane and polyvinyl chloride polymers as described in U.S. Pat. No. 4,228,194 both herein incorporated by reference in their entirety. One preferred embodiment is copper metal wire having a covering comprising a suitable electrical insulation material.

The wire can be housed inside of the smaller lumen of the catheter along some or most of the length of the device, and where it is soldered to the steel couple, it is insulated by UV curable polymer. The proximal end can be isolated from the injection port and is attached to the electrical lead of the voltage source. A one-way valve can be positioned inside the catheter lumen near the steel couple so that fluid cannot escape upon detachment. Additionally, a NITINOL coupling can be used so as to eliminate the possibility of steel emboli floating downstream.

Although several particular methods and means of detachment is disclosed herein, it is not intended to limit the invention to any particular method of detachment and any method known in the art may be used with the present invention.

An exemplary embodiment of the balloon of the invention is illustrated in FIG. 3 showing the balloon (1), the micropores (7), and the balloon lumen (8).

Polymerizing Material

The invention provides a hardening material for injecting into the balloon in the form of an adhesive fluid. The hardening material can have at least one of the following properties: the ability to secure the balloon to the aneurysm wall, such as having adhesive properties with tissue; having a low viscosity; having a curing time of minutes rather than hours; and extent of reactivity with biological fluids. Such materials are, for example, different types of cyanoacrylates, a liquid embolic polymer such as ONYX (MicroTherapeutics, Irvine Calif.), gelatin-resorcinol-formal (GRF) agents, and hydrogels of various formulations. Also considered are biological adhesive coatings such as the peptide Arg-Gly-Asp (RGD); fibrins; animal proteins, including frog or mollusk proteins; and gelatin (see for example, Silver et al. (1995) Biomaterials 16: 891; International Patent application No. PCT/AU01/01172).

Cyanoacrylates can be formulated in low viscosities and easily injected up the narrow lumen of the catheter on which the balloon is mounted. Cyanocrylate polymerizes on contact with saline or blood, allowing for fast adhesion. Cyanocrylate is effective as a tissue adhesive in both commercial as well in experimental testing.

Escape of emboli material into the parent vessel constitutes one of the potential long term short comings of aneurysm embolization with discrete GDC coils. The use of embolizing medical balloons coated with cell adhesion motifs offers a solution to this potentially fatal problem. The RGD peptide ligand is a well known adhesion motif with potential applications in this context. Numerous material surfaces used as bio-implants have been chemically functionalized or coated with the RGD peptides to induce native cell adhesion to the material surface for better biocompatibility or accelerated healing of tissue lesions.

The RGD ligand is a versatile cell recognition motif on extra cellular matrix (ECM) proteins such as fibronectin, vitronectin, and lamin. These proteins localize cell attachment to the ECM in the normal cellular environment. Most cells require attachment to survive. Of these ECM proteins, classes of fibronectin are found in virtually all physiological fluids as well as cell surfaces. Fibronectin has thus been most widely studied for their cell-adhesion properties. Like other ECM proteins, fibronectin and cells interact by the binding of cell memnbrane receptors to certain amino acid sequence (adhesion motif) in the fibronectin molecule such as RGD. Cell adhesion molecules include cadherins, selectins, immunoglobins and integrin. The integrin receptors bind with the motif and this integrin-mediated cell adhesion and signaling are crucial to, among a wide variety of processes, adhesion, cell proliferation and survival. Conversely, the loss of cell attachment leads to apoptosis (programmed cell death) in many cell types. During binding, the motif-integrin interaction induces the formation of intracellular complexes which associates actin filaments in the cell and, through a cascade of signal transductions, reorganizes the actin filaments which is known to be related to the flattened morphology associated with well attached cells. Such attachment is called focal adhesion.

The RGD sequence is by far the most effective and most often employed peptide motif for stimulated cell adhesion on synthetic surfaces. While attempting to reduce macromolecular ligands to small recognition sequences eighteen years ago, the RGD motif was identified by Pierschbacher and Rouslahti (U.S. Pat. No. 4,578,079) as a minimal essential cell adhesion peptide sequence in fibronectin. Soluble RGD peptides inhibit cell adhesion while conversely, RGD peptides immobilized upon synthetic surfaces induce cell adhesion. Also, it has been shown that about half of the integrin family of receptors bind to ECM proteins in a RGD dependent manner.

Note that although certain polymerizing materials are used in this disclosure as examples, we do not intend to limit the invention to any particular material, and any suitable polymerizing material known in the art may be used with the present invention.

Porosity Test

Rapid adhesion of the balloon to the walls of the aneurysm can be achieved by dispensing liquid adhesive through micropores of the embolizing balloon. During the embolizing procedure, it is desirable that the balloon be inflated to its desired shape and dimensions prior to dispensing the liquid emboli. In order to achieve this effect, the porosity is an important design variable.

Porosity can be measured using methods well known to those in the art, using, for example, simulating a viscous fluid with glycerol at different concentrations. The viscous fluid is used to determine the threshold pressure at which the fluid begins to be expressed through the micropore. The minimum threshold pressure will be dependant on the viscosity of the fluid being expressed. In certain embodiments the minimum threshold pressure may be 30, 60, 80, 100, 120, 140, 160, 180, 200 or 250 mm Hg. For the porosity tests, fluid temperatures can be measured at about 20° C. In the alternative, fluid temperatures can be measured at any other temperature relevant to the conditions that the operator desires. For example, the fluid temperature can be measured at 12° C., at 16° C., at 25° C., at 30° C., at 33° C., at 35° C., at 37° C., at 40° C., or at 45° C.

Subsequently, for a desired viscosity, the volume ratio (Vr) of pure glycerol to water required for the corresponding specific glycerol weight percentage is Vr=(x*ρ_{w}/ρ_{g})* (100-x) where x refers to the weight percentage and ρ_{w}, ρ_{g} refers to the density of water and pure glycerol at 20° C. respectively.

Adhesive Formulation Testing

A series of tests can be performed on an adhesive to develop an optimal formulation and delivery process for use in the balloon device and that can meet the following properties. The adhesive can be totally delivered without risk of hardening in the catheter during delivery. A non-adhesive material, such as saline, can be conducted through the catheter lumen following delivery of the adhesive so that the catheter is essentially free of uncured adhesive. The adhesive can harden to a solid mass in the balloon. A polymerization time long enough so that the operator may, if necessary, inject more saline into the balloon for several minutes following injection and delivery of adhesive. Upon contact of the adhesive with the wall of the aneurysm, results in rapid adhesion between the balloon and the wall of the aneurysm. Heating or cooling beyond physiological limits of the balloon during polymerization is less desirable but need not be excluded for the purposes of the invention.

### REFERENCE NUMBERING

1. Balloon

2. Side Hole in Double-Lumen Catheter for Extrusion of Bio-Adhesive Fluid

3. Steel Couple Element for Electrolysis

4. Double Lumen Catheter

5. GDC Detaching System

6. Non-Steel Material Couple Element

7. Micropores

8. Balloon Lumen

9. Detachable Coil (MATRIX)

10. Pusher Wire

11. Model Aneurysm

12. Model Circle of Willis

13. Model Circulatory System

14. Harvard Pump

15. Water Bath for Temperature and Physiological Equilibration

16. Electrical Potential Difference Source

17. Cathode

18. Anode

19. First Tube of Double Lumen Catheter

20. Second Tube of Double Lumen Catheter

21. TEFLON-Coated Mandrel

22. Wire (Electrically Conductive)

23. Solder Joint

24. Adhesive (UV-cured)

25. Manometer

26. Pressure Gauge Syringe

27. Four-way Connector

28. PTFE (TEFLON) Bead

### OBJECTS OF THE INVENTION

The invention addresses the following objectives:

1. The medical device must be able to prevent or minimize aneurysm rupture. Aneurysm growth can be reduced by preventing blood flow into the aneurysm, inducing thrombosis by depositing embolizing agents.

2. Medical device deployment must make use of current delivery methods. Since the project is mainly focused on the device, the delivery systems available must be compatible with device designs.

3. Medical device should be clearly monitored. This includes clear indications in visual feed back system during stages of deployment and subsequent follow up monitoring. Location of the device in the body must be detected and its orientation with respect to its desired position must be monitored.

4. Deployment time should be minimized. This is because there may be blood flow obstruction during deployment and such prolonged occlusion could cause ischemia.

5. Medical device must be permanently localized after deployment. For devices that include embolizing agents, there must be a built in mechanism that prevents movement into the parent vessel. This is to avoid breakaway debris that will cause occlusion of other vessels.

6. Medical device must have the ability to treat aneurysms as large as 10 mm neck diameter. Treatment of large wide-neck aneurysm is a medical need since such large aneurysms have the greatest risk of rupture.

7. The device may be designed for one time use only.

Physical Requirements

1. Medical device and its components should be made of biocompatible material.

2. Medical device should be easily deployed within intracranial dimensions. The deployment mechanism should be effective within these small dimensions and the device should be deployable under such conditions.

3. Catheter delivery system has length of at least 180 cm to reach the cerebrum from femoral artery access point.

4. Medical device should be compatible with catheter delivery system. Since a 2Fr internal diameter (approx. 0.012″/0.3 mm) micro-catheter is usually used to deliver devices into intracranial vessels that have diameters of approximately 2 mm or less, the device dimension must be compatible with a 2Fr lumen. Furthermore, the device should not be too tightly packed in the lumen such that it will hamper deployment.

5. Catheter stiffness is suggested for torque transmission and device control. Stiffness should be progressively softer/more flexible from proximal to distal end in order for usage through tortuous paths. The proximal end refers to the catheter end that is outside the body and the distal end refers to the tip in the intracranial vessel when fully inserted. A stiffer proximal end allows better torque control by the surgeon that will help the catheter navigate through tortuous blood vessel anatomy.

6. Device should withstand sharp turns and tortuous navigation paths. Since the catheter delivery system as well as the device must pass through the carotid artery that is often tortuous (especially true for older patients) before reaching the cerebral aneurysms. Furthermore the intracranial vessels have differing degrees of tortuosity.

7. Medical device conforms to geometry of bifurcation. It should fit well within the bifurcation to provide proper placement.

8. Device should be robustly attached to catheter deployment system to prevent in vivo breakage failure. This is because forces experienced by the device within the delivery lumen could be significant during navigation through tortuous pathways.

9. The design should incorporate radiopaque markers to show the distal end of the catheter delivery system as well as the device position for monitoring during surgery

10. There should be allowance for device retrieval (but will not be necessary if the device has already been deployed).

11. Proper electric isolation and circuit design should be considered for use in the deployment mechanism. Use of low voltage DC is employed to minimize risk to the patient.

Design Constraints

1. Treatment should not cause rupture of aneurysm.

2. Treatment should not concentrate stress in one location. Loading in aneurysm should be distributed as much as possible since stress concentration can cause post-operative perfusion.

3. Treatment should not concentrate stress in one direction. Aneurysm walls are largely anisotropic being weaker in the “latitudinal” direction compared to the “longitudinal” direction. Disproportionate loading in the weak direction can accelerate ruptures. Literature studies indicate aneurysm wall strength to be about 0.5 MPa±0.25 MPa. The lower limit of this range is taken for safety purposes.

4. Deployment pressure should not exceed 150 mmHg.

5. Medical device deployment should not be too complex. This is because the deployment mechanism should fit within the small delivery lumen. Consequently, deployment procedures should be simplified and reliable.

6. Device should not take an excessive amount of time to deploy. As previously mentioned, prolonged deployment time will increase the risk of device failure, and negative biological response.

7. The medical device should not cause an adverse immune reaction in the patient or be harmful to patient.

8. Medical device should minimize lacerations to vessel walls during delivery. Vessel wall injury during catheter navigation is an issue and this is especially important in tortuous vessels where sharp turns in the anatomy could create significant friction forces between the vessel walls and the delivery catheter.

### OBJECTS AND ADVANTAGES OF AN EXEMPLARY INVENTION

The key points of the invention design can be summed up as follows:

Reduced Risk of Rupture Due to Stress Concentration

From background research, it was discovered that the weakest point in the aneurysm geometry is the apex of the protrusion, which is also the site of rupture in the majority of cases. The device of the ivnetion is designed to minimize the total amount of stress exerted on the aneurysm wall required to ensure placement of the device and to divert the stress concentration points away from the apex of the aneurysm.

Balloon Adhesion to Vessel Wall

Porous balloon with cyanoacrylate—Using a porous balloon in conjunction with a cyanoacrylate adhesive to inflate and embolize the balloon is a means to apply adhesive to the outside of the balloon, allowing the device to physically bond to the aneurysm wall and thereby preventing migration or dislodging.

Balloon Embolization with Glue

The device uses a hardening polymer such as cyanoacrylate within the balloon in order to create a solid mass after deployment. Because there is the potential for the balloon to wear over time and potentially rupture, the use of the polymer instead of saline is an attractive feature because it will ensure the safety of the device for long-term placement. If the balloon were filled with saline, there is the possibility that after rupture, the balloon remnants could dislodge from its position to occlude the parent vessel. By forming a solid mass, the device will remain in place even if a hole in the balloon wall forms.

Safe Detachment System

Currently, detachable endovascular balloons employed in cases of vascular reconstruction are deployed by pulling on the catheter to break the connection between catheter and device. Compared to the traditional method of pulling on the catheter, the use of a non-forceful method of detachment, such as an electrically controlled balloon deployment, is advantageous in the treatment of aneurysms because it reduces the risk of movement of the balloon after placement as well as stress on the delicate cerebral vasculature.

Safety

Retractable design—Since permanent polymer embolization for the device is to be so used, a deployment process is disclosed which enables the physician to first check the balloon size and fit using saline prior to the introduction of the polymer. Therefore, if the balloon is found to be unsuitable for the aneurysm geometry, the physician can remove the device without harm to the patient.

Simple Operation

By basing the operation of our device on already existing endovascular techniques, training time for a physician or an operator is minimized to learn how to use our device, which can increase the speed of adoption. Using existing techniques also minimizes the risks associated with designing completely novel methods that may take more time to fine-tune and require extensive feedback testing from end users.

Fast Device Deployment

Compared to coiling techniques that require the deployment of several devices before the aneurysm can be successfully occluded, our device is such that only a single unit is required in order to occlude the target aneurysm. The time-savings is beneficial to both the patient, who can be anesthetized for shorter periods of time, and the physician, who will experience less stress and fatigue from long, tedious operations.

Range of Application

While the device was designed specifically to treat wide-neck aneurysms, it can also be easily adapted for narrow neck aneurysm geometry.

Additionally, a failure mode and effect analysis (FMEA) was performed on our final device design. The results of the FMEA shows that the risks associated with our final design are such that we can be justified in continuing this project.

The invention will be more readily understood by reference to the following examples, which are included merely for purposes of illustration of certain aspects and embodiments of the present invention and not as limitations.

### EXAMPLES

### Example I

### Aneurysm Cast Making for Testing Balloon

Blow Molding

In order to create our in vitro test platform for the deployment of our device, we utilized a blow molding process. Using this blow molding process, we were able to create soft, compliant models of the aneurysm.

The basic idea behind this blow molding process is to first heat a section of vinyl tubing until soft, and then introduce compressed air into the tubing to expand and permanently deform the shape. If the tubing is constrained within a TEFLON mold, the tubing will expand to the form of the mold when compressed air is introduced.

The tubing can be heated one of two ways: through direct heating of the outer tubing surface or heating through the interior pathway. Points exposed to greater heat will expand more than its surrounding area, therefore, direct heating of the outer surface can be used to make arbitrary shapes by manipulating the localized heating of the tubing. On the other hand, while heating through the tubing pathway takes more time, the result is a very uniform tubing expansion.

Materials

| | |
|---|---|
| Vinyl tubing | Hose clamps and barbs |
| Regulator | 3 way ball valve |
| Compressed air supply | Vise grips |
| Hot air supply (hot air gun w/temperature | Teflon mold |
| controller) | |

Method

1. Check that the regulator is off and the ball valve is turned to the proper position. Secure a piece of tubing to the hose barb with a hose clamp.

2. Heat the tubing until soft.

3. Clamp tubing end and turn ball valve to the compressed air line side.

4. Slowly increase the pressure within the tubing by adjusting the regulator.

5. Allow the tubing to cool while still pressurized.

6. Release pressure by adjusting the regulator and remove tubing.

Bifurcation Model

With the aneurysm models created from the blow molding process, in vitro circuit models were completed by using hot glue or silicone adhesive to affix the models to additional tubing pieces in order to simulate bifurcation geometry.

### Example II

### Balloon Manufacture

A urethane balloon of 12 mm diameter was made through blow molding or vacuum forming with appropriate glass casts while the double lumen catheter was manufactured by extrusion of clear PEBAX resins through an extrusion machine. However, the test device was assembled manually.

Flourinated ethylene propylene (FEP) heat shrink tubing was used to bond the urethane balloon to the double lumen catheter. When heat was applied to the tubing, the shrinking of the tubing as well as the thermal molecular compatibility of the urethane and PEBAX allowed a strong bond to form between the two materials. The test device consisted of a double lumen catheter with a detachable distal section adjoined via a stainless steel coupling to the rest of the catheter (see FIG. 13). Hence, the assembly of the device could be separated into two portions as follows.

Detachable Distal End

The distal end was manufactured as follows:

1. The collars of the urethane balloon were first trimmed.

2. A TEFLON (PTFE) bead (28) of 44×0.001″ diameter was passed into the larger lumen of the catheter while a 14×0.001″ diameter TEFLON coated mandrel (21) was passed into the smaller lumen.

3. An opening into the larger lumen was carved out with the use of a razor blade. When the device is assembled, this opening allows the embolic fluid to fill up the balloon lumen.

4. The urethane balloon was placed over the double lumen catheter as shown in FIG. 13 and heat shrink tubing was placed over the balloon collars.

5. The heating arm of a hot box was used to heat the heat shrink tubing to about 420° F. However, prior to heating, the urethane balloon was shielded with a flexible silicone rubber collar to protect the balloon from the heat.

6. After the tubing sections were heat shrunk, compressed air was used to cool the tubing so that the heat-shrunk tubing could be carefully peeled off.

7. Finally, the PTFE bead and TEFLON-coated mandrel were carefully pulled out.

Main Device Length

The second portion of the testing device consisted of the main length of the double lumen catheter with a coupling end on which the detachable distal end were attached (see FIG. 14). Double lumen catheters used for manufacturing both the distal end and main device length were made from clear PEBAX resin.

Steps to assemble the coupling end were as follows:

1. A length of copper wire was first soldered onto the stainless steel coupling using an acid-based flux.

2. Both the stainless steel coupling and the copper wire were then fitted into their respective lumens by first feeding the copper wire into its lumen until it passed out the other end of the catheter. While passing the cooper wire into its lumen, the steel coupling was eventually fitted into its designated lumen.

3. In preparation for bonding of the steel coupling to the double lumen catheter, the lower end of the steel coupling was coated with a small amount of UV curable adhesive. Next, the adhesive is exposed to UV light to set the adhesive and form a permanent bond.

4. Finally, in order to insulate the solder joint between the cooper wire and steel coupling, UV cured adhesive was used to coat the joint. FIG. 14 shows the final assembly of the coupling end.

5. In the final steps before total assembly, the top end of the detachable section was sealed with silicone. Finally, the distal detachable end was bonded to the steel coupling with UV curable adhesive. During in vitro testing, the proximal end of the test device would be coupled to a connector to allow infusion of the test emboli.

6. In order to interface syringes to our device, a luered Y-connector was bonded to the proximal end of the device with UV adhesive. The copper lead was first fed out through the side port so that the catheter could be bonded to the connector. Next, a small bit of tubing was passed over the copper wire and bonded into the side port with UV adhesive. The tubing section was necessary to provide strain relief for the copper wire and prevents kinking or breakage. Finally the tubing end was sealed with more UV adhesive to prevent leakage.

### Example III

### In Vitro Testing

(i) Test Setup

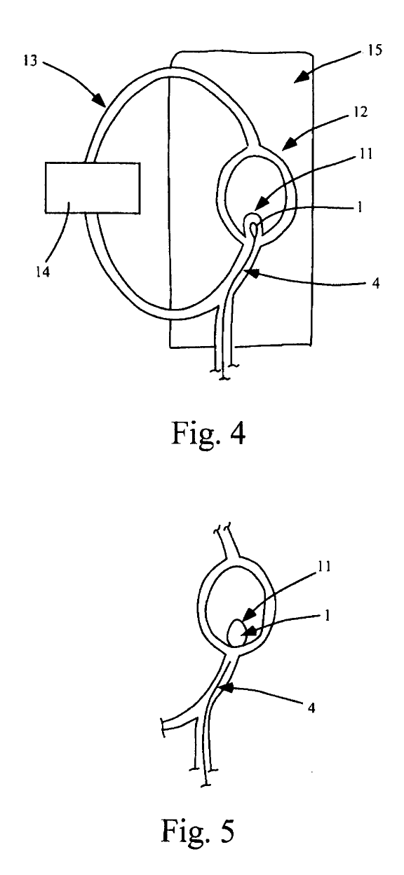

In vitro tests for proof-of-concept devices were performed for prototypes of scale 5× and 2×. The setup for the in vitro test is shown in FIG. 4. Tests were performed in simplified intracranial vasculature phantoms manufactured to corresponding scales from heat-treated PVC tubing as describe in Example I.

The model vasculature in FIG. 4 consists of a simplified Circle of Willis replica (12) with a wide neck aneurysm at the bifurcation of the basilar artery (11). The vasculature phantom was placed in a water bath (15) of phosphate buffered saline solution mimicking the pH and electrical properties of blood. Hemodynamic conditions were simulated by driving a pulsatile-flow with a Harvard pump (14). As shown, the test circuit includes the phantom vasculature and the bath. An entry point in the vasculature phantom allows the test device to be inserted and delivered to the aneurysm site.

(ii) In Vitro Test at 5× Scale

In the 5× scale test, the test device was a PEBAX catheter mounted with a low durometer urethane balloon purchased from Advanced Polymers. Pores of about 10 μm diameter were cut by hand into the balloon material. The test was performed in an appropriately scaled intracranial vasculature phantom that included an aneurysm with a neck size of approximately 1.3 cm. Table 1 shows experimental conditions for the in vitro test.

TABLE 1
Experimental Conditions for 5 × scaled test

| Test Device Specifications | | Liquid Emboli Specifications | | Harvard Pump | | |
|---|---|---|---|---|---|---|
| | | | | Harvard Apparatus | | |
| | | | | Model 1421 | | |
| (a) Porous Balloon | | (a) Cyanoacrylate | | (a) Settings | | |
| Low durometer urethane | | Loctite 4014 Ethyl cyanoacrylate | | Stroke volume: | 9.7 | cc |
| Balloon Diam: | 30 mm | Viscosity: | 3 cP | Percent Systole: | 55 | |
| Pore Diam: | 10 um | | | Pump rate: | 79 | rpm |

| (b) Catheter | (b) Retardent | |
|---|---|---|
| Pebax | Acetic acid (more than 80% by mass) | |
| Outer Diam: 0.063 in | Emboli to retardent | |
| | ration by volume: | 1 |

| | |
|---|---|
| (c) Steel Detachment Coupling | (c) Saline |
| 316 Stainless Steel | Phosphate Buffered Saline |

| | | |
|---|---|---|
| Outer Diam: | 0.042 | in |
| Length: | 8.00 | mm |

(ii)(a) Test Procedure

PBS solution was prepared as the electrolyte solution. The Harvard pump was switched on and adjusted to the parameters shown in Table 1. The pulsatile flow was allowed to circulate through the vasculature phantom for fifteen minutes in order to prime the flow circuit and drive out any air bubbles within the PVC tubing.

The liquid emboli components were prepared for use as follows. The volume of the inflated balloon was estimated by measuring the volume displaced in the injection syringe while inflating the collapsed balloon. A volume of cyanoacrylate emboli equal to the volume of the balloon and a slightly larger volume of saline was prepared. Acetic acid was then added into the saline at 2 drops per 30 ml. During liquid emboli injection, saline was used to chase cyanoacrylate and subsequent mixing of cyanoacrylate, saline, and acetic acid as a retardant resulted in a slowly curing occlusion at the aneurysm neck. Composition ratios for the liquid emboli are shown in Table 1. The slightly larger volume of saline accounts for the volume of the catheter lumen.

With the test balloon deflated, the catheter (4) was inserted into the vasculature phantom at the entry point as shown in FIG. 4.

The catheter was guided through the vasculature to the wide neck aneurysm situated at the bifurcation of the phantom model.

To simulate the surgical implementation procedures, the porous balloon was first inflated with saline to determine if the inflated size was suitable for the wide neck aneurysm, after which the saline was withdrawn in preparation for actual delivery of the liquid emboli.

Cyanoacrylate and the saline solution prepared above was injected via the catheter into the porous balloon with the use of two adapted syringes coupled to a connector at the proximal end of the catheter. A stopwatch was immediately started to track the time required for secure attachment of the porous balloon to the aneurysm wall and the time required for the curing of the cyanoacrylate emboli in the balloon lumen.

As the porous balloon became fully inflated, a slight additional pressure was applied to dispense a coating of cyanoacrylate through the pores. (See FIG. 5.)

(ii)(b) Results and Conclusion

After approximately one minute, the balloon was assessed to be securely attached to the walls of the aneurysm phantom by pulling on the catheter. The cyanoacrylate emboli in the balloon was observed to start to cure and harden after three minutes. FIG. 5 shows the hardened porous balloon inside the aneurysm phantom. This took about 12 hours. The tensile force required to disengage the balloon from aneurysm wall was measured at newtons (N) with a force gauge. This greatly exceeds physiological forces the balloon would be expected to encounter.

A very tiny amount of cyanoacrylate was observed escaping from the aneurysm into the vasculature phantom when the cyanoacrylate emboli was dispensed out of the balloon pores. This could be attributed to the fact that the balloon pores are not microporous and the dispensed liquid was not sufficiently localized. We predict that the use of microporous material in further iterations of the design will prevent such leakage as previously discussed.

FIG. 5 shows that the aneurysm phantom was well occluded during balloon inflation and cyanoacrylate curing. In addition, the aneurysm did not feel significantly warmer during the cyanoacrylate curing. Hence, there was little resultant temperature rise due to the exothermic reaction of cyanoacrylate polymerization.

(iii) In Vitro Test at 2× Scale

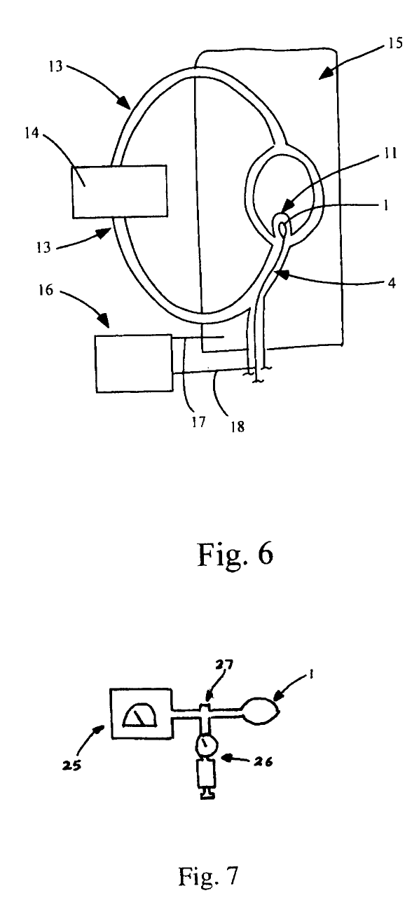

Using a similar experimental setup shown in FIG. 6, the 2× scale in vitro test was performed using a corresponding sized vasculature phantom with an aneurysm neck size of 7 mm. Since balloon detachment testing was an important objective of this in vitro setup, the test device consists of a 12 mm diameter porous balloon mounted to the distal end of a double lumen catheter with a detachable steel coupling. As detailed in the description of our design, liquid emboli was transported through the main lumen of the catheter and the peripheral lumen served as an insulating conduit which housed the electrolytic detachment wire soldered to the stainless steel coupling. Ultraviolet curable adhesive was further used to encase the exposed solder joint. Both the balloon and the double lumen catheter were manufactured in an off-campus corporate facility and the corresponding methods are shown in Example II.

FIG. 6 shows the experimental setup for the 2× scale in vitro test. In addition to the Harvard pump (14) and vasculature phantom (11), the proximal end of the electrolytic copper wire (18) was connected to the negative terminal of a voltage source to act as the anode. The positive terminal (17) of the voltage source was immersed into the saline to act as the cathode. Such an arrangement simulates the setup for electrolytic detachment used in common occluding device. To check for any possible leaking of polymer from the balloon into fluid stream, filters were made out of cloth bandages and placed distal to the aneurysm and proximal to the inlet of the pump. Table 2 shows the experimental conditions for this test.

TABLE 2
Experimental Conditions for 2 × times scaled test

| Test Device Specifications | | | Liquid Emboli Specifications | | | Harvard Pump | | | Voltage Generator/Power Supply | |
|---|---|---|---|---|---|---|---|---|---|---|
| | | | | | | Harvard Apparatus | | | BK Precision | |
| | | | | | | Model 1421 | | | Model 1621 A | |
| (a) Porous Balloon | | | (a) Cyanoacrylate | | | (a) Settings | | | (a) Settings | |
| Low durometer urethane | | | Loctite 4014 Ethyl cyanoacrylate | | | Stroke volume: | 9.7 | cc | Voltage: | 18 V |
| Balloon Diam: | 8 | mm | Viscosity: | 3 | cP | Percent Systole: | 55 | | Current: | 30 mA-70 mA |
| Pore Diam: | 0.004 | in | | | | Pump rate: | 79 | rpm | | |

| (b) Double Lumen Catheter | | (b) Retardent | |
|---|---|---|---|
| Pebax | | Acetic acid (more than 80% by mass) | |
| Outer Diam: | 0.063 in | Emboli to retardent | |
| Inner Diam 1: | 50 × 0.001 in | ration by volume: | 1 |
| Inner Diam 2: | 16 × 0.001 in | | |

| | |
|---|---|
| (c) Steel Detachment Coupling | (c) Saline |
| 316 Stainless Steel | Phosphate Buffered Saline |

| | | |
|---|---|---|
| Outer Diam: | 0.042 | in |
| Length: | 8.00 | mm |

(d) Electrolytic Wire
Copper

| | | |
|---|---|---|
| Diam: | 0.01″ | in |

(iii)(a) Test Procedure

PBS solution was prepared. The Harvard pump was switched on and adjusted to the parameters shown in Table 2. The pulsatile flow was allowed to circulate through the vasculature phantom for 15 minutes in order to prime the flow circuit and drive out any air bubbles within the PVC tubing.

The liquid emboli components were prepared for use as follows. The volume of the inflated balloon was estimated by measuring the volume displaced in the injection syringe while inflating the collapsed balloon. An equal volume of cyanoacrylate emboli and a slightly larger amount of saline was prepared. Acetic acid was then added into the saline at 2 drops per 30 ml. During liquid emboli injection, saline was used to chase cyanoacrylate and subsequent mixing of cyanoacrylate and saline with acetic acid as a retardant allowed a controllable hardening process. Composition ratios for the liquid emboli are shown in Table 2. The slightly larger volume of saline accounts for the volume of the catheter lumen.

With the test balloon deflated, the catheter was inserted into the vasculature phantom at the entry point as shown in FIG. 6.

The catheter was guided through the vasculature to the wide neck aneurysm situated at the bifurcation of the phantom model.

To simulate the surgical procedure, the porous balloon was first inflated with saline to determine if the inflated size was suitable for the wide neck aneurysm, after which the saline was withdrawn in preparation for actual delivery of the liquid emboli.

Cyanoacrylate and the saline prepared as described above were injected via the catheter into the porous balloon with the use of two adapted syringes coupled to a connector at the proximal end of the catheter. A stopwatch was started to track the time required for secure attachment of the porous balloon to the aneurysm wall and the time required for the curing of the cyanoacrylate emboli in the balloon lumen.

As the porous balloon became fully inflated, a slight additional pressure was applied to dispense a coating of cyanoacrylate through the pores.

After allowing 2-3 minutes for secure balloon adhesion, the voltage generator was switched on to initiate electrolytic detachment. A second timer was started. Time for complete electrolytic detachment was recorded.

(iii)(b) Results and Conclusion

Because of the short catheter length of our prototype device the positioning of our device was very sensitive to movements at the proximal end. During the introduction of the cyanoacrylate into the balloon, the catheter was accidentally bumped, causing the balloon device to partially slip out of the aneurysm and partially block the fluid pathway of the parent vessel as it began to cure. Despite the slippage, because the micropores were confined to the upper regions of the device no cyanoacrylate was noticed leaking out of the aneurysm and escaping downstream. This is an advantage of the current invention over the prior art. Earlier tests of polymer embolization demonstrated that when small droplets of glue come into contact with a fluid stream, the droplets cured into large, easily visible chunks of foamy polymer. Subsequent inspection of the emboli filters placed downstream confirmed this observation, as nothing was found in the traps. Additionally, the device was still capable of maintaining good adhesion to the aneurysm wall, with adhesion occurring within about one minute after injection of cyanoacrylate and saline. While the matter of the device slipping may be an issue of concern for ease of use, we feel that this problem can be solved through the catheter design.

Approximately five minutes was required to electrolytically detach the device from its delivery catheter. However, because of the insulating nature of the vinyl tubing model of the vasculature, we encountered some initial difficulty in generating enough current to perform the electrolysis. While very little current was generated at the onset of electrolysis, as the ground wire was moved closer to the steel couple, the amount of current passing through the leads also increased until the ground wire was about 1″ away from the couple with a 70 mA output. Despite this problem related to the device release, it is not expected to be clinically significant since tissue and blood conduct electricity well compared to the materials in our experimental setup.

After the device was released, the model was removed from the electrolyte reservoir for inspection. Visual inspection of the balloon showed that there were some wrinkles in the balloon material which allowed some fluid to pass through the sealing perimeter when the bulb end of the aneurysm model was squeezed. These wrinkles are due to the fact that the balloon is intended to be slightly oversized compared to the aneurysm, but it is not a severe issue because the device was still firmly attached to the walls of the model and sufficiently blocking flow to the aneurysm. Additionally, as the porosity of our device improves, the overall cyanoacrylate coverage of the device will increase over our and made models thereby making it possible that the any such wrinkles will seal themselves off.

Overall, the results from the testing of the 2× scale prototype successfully mirrored results from previous testing of devices on the larger scales and proved the efficacy of the concept that we have developed in occluding aneurysms.

### Example IV

### Porosity of Test Balloons

Three microporous test balloons were purchased from Advanced Polymers and used to correlate porosity properties with dispensing pressures. In addition to porosity parameters, the effects of microporosity is anticipated to depend on liquid emboli viscosity since fluid surface tension could affect threshold pressures. For porosity validation and evaluation, threshold pressures for fluids of different viscosities were measured. Polymerizing fluids of differing viscosities were simulated by adjusting glycerol weight percentage in aqueous glycerol solutions. Variation of viscosities for different compositions of aqueous glycerol at various temperatures can be found in standard chemistry handbooks, such as the “CRC Handbook of Chemistry and Physics, 85^{th} edition, 2004-2005” (CRC Press, Boca Raton Fla.).

TABLE 3
Aqueous glycerol composition at
varying viscosities

| fluid # | fluid composition | viscosity (cP) |
|---|---|---|
| 1 | water | 1.005 |
| 2 | 10% glycerol | 1.31 |
| 3 | 20% glycerol | 1.76 |
| 4 | 30% glycerol | 2.5 |
| 5 | 40% glycerol | 3.72 |

TABLE 4
Manufacturer's specifications

| | pore | | | % open | open area |
|---|---|---|---|---|---|
| balloon | size (cm) | pore density | surface area | area | (cm\hat{ }2) |
| A | 6.00E−05 | 3.70E+05 | 2.513272 | 0.42% | 2.63E−03 |
| B | 5.90E−05 | 2.00E+06 | 2.513272 | 2.19% | 1.37E−02 |
| C | 4.60E−05 | 2.30E+06 | 2.513272 | 1.53% | 9.61E−03 |

TABLE 5
Test data and linear parameters

| Balloon A | pressure | Balloon B | pressure | Balloon C | pressure |
|---|---|---|---|---|---|
| viscosity (cP) | (mm Hg) | viscosity (cP) | (mm Hg) | viscosity (cP) | (mm Hg) |
| 1.005 | 1025 | 1.005 | 220 | 1.005 | 290 |
| 1.31 | 915 | 1.31 | 290 | 1.31 | 450 |
| 1.76 | 1093 | 1.76 | 500 | 1.76 | 865 |
| 2.5 | 880 | 2.5 | 680 | 2.5 | 810 |
| 3.72 | 740 | 3.72 | 760 | 3.72 | 1100 |
| | | m | 204.3917772 | m | 272.119023 |
| | | b | 69.15733066 | b | 142.7069317 |
| | | normalized m | 2.81E+00 | normalized m | 2.61E+00 |
| | | for open area | | for open area | |

The balloons (1) were placed on the end of a syringe (26) and four-way connector (27) with one end going to a digital manometer (25) as is shown in FIG. 7. The balloons were first inflated and then pressurized further to force fluid out of the micropores. In this manner, the threshold pressures of each balloon were measured for each fluid viscosity.

For the porosity tests, fluid temperatures were measured at about 20° C. Subsequently, for a desired viscosity, the volume ratio of pure glycerol to water required for the corresponding specific glycerol weight percentage, Vr=(x*ρ_{w}/ρ_{g})*(100-x) where x refers to the weight percentage and ρ_{w}, ρ_{g} refers to the density of water and pure glycerol at 20° C. respectively.

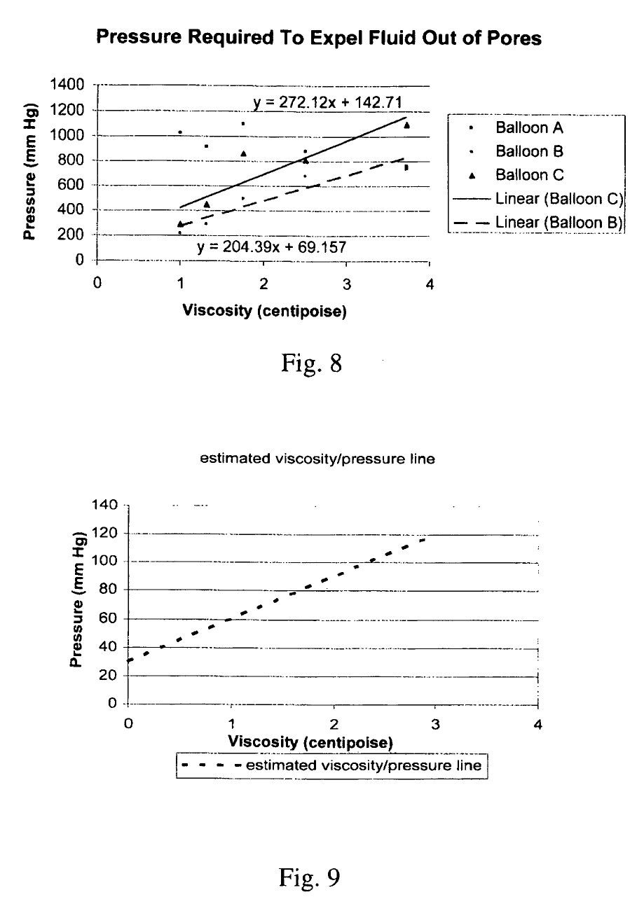

Table 3 shows fluid viscosity used in the tests and their corresponding glycerol compositions. For the three microporous balloon used, Table 4 shows manufacturer specifications for each balloon as well as calculated open areas. Table 5 shows experimental results of threshold pressures for a set of fluid viscosities tested on each balloon and the parameters of gradient and intercept associated with estimated linear correlation of experimental data. FIG. 8 shows experimental data with their estimated linear trend. A linear trend line was not estimated for balloon A because experimental data was inconsistent and the measured pressures were far out of our target range. Hence, only the data from balloon B and balloon C was used for this study.

From observations, the product of the linear slope and pore open areas could be estimated as a constant for both balloon B and balloon C. This constant has an average value of 2.71 mm Hg*cm²/cp. For the dimensions of the current balloon design, Table 6 and FIG. 9 show the targeted manufacturing parameters obtained using the estimated constant, anticipated glue viscosity, physiological pressure conditions, desired threshold pressure and calculated balloon surface area. Consequently, outsourced balloons required for this application would have a 2% open area with threshold pressures ranging from 30 mm Hg to 120 mm Hg. The minimum threshold pressure will be dependant on the viscosity of the fluid being expressed. In certain embodiments the minimum threshold pressure may be 30, 60, 80, 100, 120, 140, 160, 180, 200 or 250 mm Hg.

TABLE 6
Design variables and targeted parameters

| Calculations | |
|---|---|
| average normalized mn | 2.711495932 |
| target pressure P(mm Hg) | 120 |
| estimated b (mm Hg) | 30 |
| glue viscosity u (cP) | 3 |
| target open area (cm\hat{ }2) | 0.090383198 |
| estimated balloon area | 4.5238896 |
| target % open area | 2.00% |

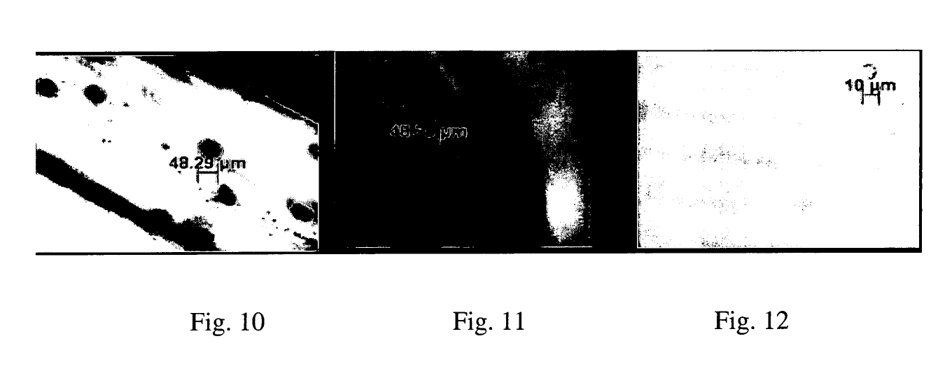

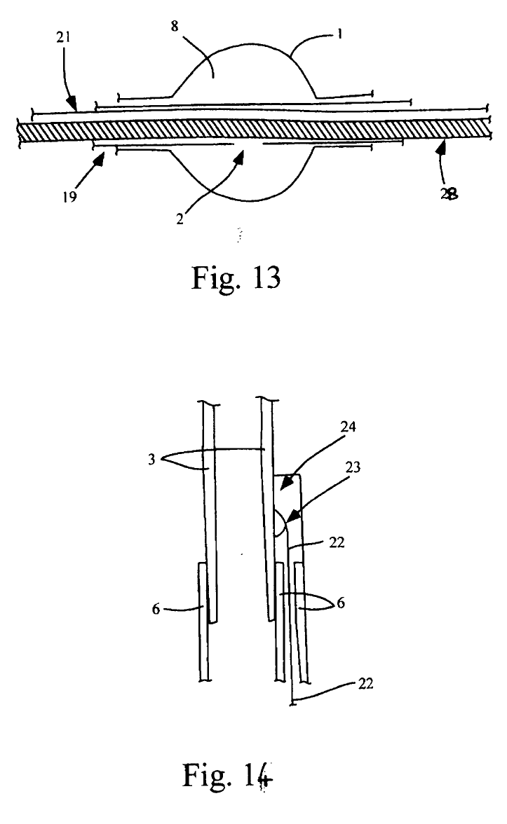

In this example, pores were made in-house using available university equipment to approximate geometries of the pores in device prototypes. FIGS. 10, 11, and 12 show magnified images of handmade pores in balloon material. The test pores are made with (from left to right) a sewing needle tip, a hypodermic needle tip, and a fine wire and are cut into the upper hemisphere of test balloons. The sewing needle and the hypodermic needle tip were too large to keep the liquid adhering to the surface of the balloon (see FIG. 10 and FIG. 11). Tests indicated a pore diameter of 10 μm is tolerable and FIG. 12 shows liquid emboli localized on a test balloon surface for such a pore size. These pores proved suitable for testing purposes despite being larger than commercially manufactured balloon pores.

From close observations, liquid emboli dispensed through micropores remained localized and coated the balloon surface well. The liquid emboli layer on the surface consists of minute droplets that do not aggregate and form large droplets that may dislocate due to balloon agitation. Such a property is of use for the actual application of the embolizing balloon since it keeps the dispensed emboli close to the balloon surface and prevents it from leaking out into the parent vessel. Note the localization of emboli to the upper portion of the balloon.

Although further work can be done to improve the formulation, from our research efforts we have found the optimal glue formulation to be a 1:1 ratio of cyanoacrylate to a mixture of 15 ml saline and 1 drop (approximately 25-50 μl) of glacial acetic acid. All subsequent testing with our devices were performed using this solution unless otherwise noted.

### Example V

### Ex Vivo Glue Adherence Test

To confirm the ability of the balloon to bind to biological tissue, an ex vivo test was developed using a cow heart. A mixture of 8 ml cyanoacrylate with 16 drops acid was used to lower the pH of the overall solution. A slightly oversized porous balloon (20-30% larger radially when inflated than its target periphery) was directed on a catheter into the mitral valve of the heart and inflated with saline to check for correct sizing. Subsequently, after removing the saline, the glue formulation was injected into the balloon and chased with saline. After 1 minute, the balloon had adhered to the tissue. After an hour, using a force gauge, the force required to pull the out the balloon out of the atrium was measured at 10 N. Calculations estimating the force exerted radially on the same balloon by the blood flow in the carotid artery would be about 1.3 N. Thus, the shear strength of the adhesive exceeded estimated physiological limits.

### Example VI

### Glue Formulation Testing

Objectives

The purpose of the glue formulation is to provide adhesion between the balloon and the aneurysmal wall. The strongest adhesive, where several of its derivatives have gained FDA approval for use in the human body, is cyanoacrylate. Currently, polymers used for embolization of cerebral aneurysms, such as ONYX and hydrogels, only exhibit cohesive properties. Thus, because of its adhesive properties and minimized regulatory concerns, cyanoacrylate was chosen as the basis for our glue formulation.

Beyond achieving its adhesive purpose, the glue formulation also needed to fit within the realm our design constraints and requirements. From careful consideration and analysis of these constraints, device requirements, and the working environment of the glue formulation, a set of requirements and constraints specific to the glue formulation were drawn out.

Background research has shown that cyanoacrylate will polymerize when in contact with anionic material or solution. Thus, blood, which is slightly basic with a pH of 7.4, may trigger the polymerization of cyanoacrylate. Cyanoacrylate may also polymerize upon contact with surfaces carrying a net negative charge, such as plastics and wood. Our deployment design required that contrast solution be used to check the size and fit of the balloon in the aneurysm prior to filling it with glue. Thus, when introducing pure cyanoacrylate into the catheter lumen after checking it with contrast, two potential activators of cyanoacrylate polymerization during its delivery exist: any residual contrast solution (since its pH is similar to that of blood) left behind in the catheter lumen and the surface contact with the catheter material.

Such premature activation of polymerization during delivery causes concern that pure cyanoacrylate could cure in the catheter lumen during its delivery. A series of tests were performed to demonstrate this concept. Catheter tubes were connected at one end to solid rubber balloons. After injecting and withdrawing saline from the balloon, it was found that subsequent injection of a volume of cyanoacrylate into the balloon could not be completed. The cyanoacrylate would polymerize within the catheter line prior to even reaching the balloon. Thus, methods needed to be developed to retard or offset the polymerization of cyanoacrylate so that the danger of an occluded catheter would be minimized.

Associated with the polymerization of cyanoacrylate is its exothermic reaction. Background research has shown that TRUFILL, an FDA approved cyanoacrylate-based medical glue used to embolize cerebral arteriovenous malformations, takes advantage of the polymer's exothermic reaction and consequential heating to apoptose the target vessel. Thus, the glue would need to be formulated such that the resulting heating from polymerization could be minimized and not cause harm to the aneurysmal wall.

Because a balloon may degrade over time in an aneurysm, a polymer that can harden to form a solid plug inside the aneurysm is required. Where a fluid filled a balloon persists in an aneurysm, rupture of the balloon could cause for migration of emboli downstream, thus causing for risk of stroke. A glue formulation that could over time harden inside the balloon would achieve this requirement.

Choice of Cyanoacrylate

Background research has shown that cyanoacrylates associated with smaller functional groups, such as methyl cyanoacrylate (for example, SUPERGLUE), form stronger bonds, but are also more toxic to biological tissue than larger functional group cyanoacrylates. The toxicity arises from the breakdown products of the polymerization reaction: cyanoacetate and formaldehyde, which can cause inflammatory reactions⁵. Larger functional group cyanoacrylates, like octyl cyanoacrylates (for example, LIQUID BANDAGE by Johnson & Johnson, New Brunswick N.J.), despite forming weaker bonds, are often used in wound care (for example, used for binding opposing edges of cuts).

Currently, the FDA cyanoacrylate with a neuro-based application is n-butyl cyanoacrylate (such as TRUFILL by Cordis Neurovascular, Inc., Miami Lakes Fla.). Ideally, our group would have preferred to utilize this cyanoacrylate. However, Cordis was unable to provide us with sufficient quantities of their product, as demanded by the testing phase of the design process. A consideration that was made was to utilize a more readily available “off-the-shelf” cyanoacrylate based product. LOCTITE offered low viscosity ethyl cyanoacrylates. These cyanoacrylates, however perhaps being more toxic than TRUFILL or LIQUID BANDAGE, can presumably form stronger bonds between the balloon and the aneurysmal wall. Since much of the cells comprising the aneurysmal wall have already naturally reached apoptosis, the greater toxicity of ethyl cyanoacrylate as opposed to octyl cyanoacrylate may not have a significant detrimental impact on the aneurysmal wall.

At the end, the objective of this project is a proof of concept model, where long-term clinical studies are not possible. Since the short and long term effects of the adhesive on the biological tissue and its associated biochemical processes cannot be studied in the time span of developing this proof of concept model, selection of a cyanoacrylate shall be primarily based on its mechanical properties. For these reasons, LOCTITE 4014, an ethyl cyanoacrylate, was chosen primarily because its markedly low viscosity (average of 3 centipoise) reduces the effects of shear stress upon delivery through the catheter, while providing for relatively strong adhesive properties.

Glue Activator Tests

To properly address these requirements for the glue formulation, a preliminary set of tests were devised to investigate different characteristics of cyanoacrylate polymerization with varied amounts of phosphate buffered saline (PBS). Four different containers were setup each with 1 ml of cyanoacrylate (CA) and different amounts of saline. Four different variables were measured for with each mixture of CA and saline: polymerization time, nature of cured polymer and the maximum temperature achieved during polymerization. The following table illustrates the results of this test:

TABLE 7
Polymerization matrix with saline

| | Mixture with 1 ml CA | | | |
|---|---|---|---|---|
| | 0.25 ml | 0.5 ml | 0.75 ml | 1 ml |
| | saline | saline | saline | saline |
| Polymerization | 2 | 3.5 | 4.3 | 5 |
| time (min) | | | | |
| Nature of cured | Hard | Hard | Hard foam, | Hard foam, |
| polymer | | | excess fluid | excess fluid |
| Max temp. (° C.) | 36 | 42 | 37 | 35 |

Despite the reasonably long polymerization times for all these mixtures, subsequent tests in catheters connected at one end to solid rubber balloons showed that the polymer cured in the catheter line during injection. Such an observation can be rationalized by taking into account the relatively large surface contact between polymer and plastic surface in the catheter lumen (as previously explained), which is of much less of significance in these tests. Some mixtures demonstrated large degrees of heating with temperatures beyond physiological, normal blood temperature. In reality, the recorded temperatures were less than the actual temperature of the polymer because of a protective plastic shield, which was placed over the thermometer end during temperature reading. Thus, the temperatures of all these mixtures may have rose above physiological, normal blood temperature. It was demonstrated that CA can react with only a certain concentration of saline. Any saline in excess of this threshold would be left behind after polymerization. Conversely, other tests showed that too little saline (anything below 0.25 ml saline with 1 ml CA) would not allow for polymerization of all the CA in the mixture. Such findings were confirmed in later tests in catheters connected to nonporous latex balloons, where it was found that the injected CA without any saline did not cure at all. Therefore, these tests demonstrated an existence of a bandwidth of the amount of activator to be used with cyanoacrylate for proper curing.

Glue Retardant Tests

Background research was performed to find methods of retarding polymerization of cyanoacrylate. Three different retardants were found: ethiodized oil (for example, poppy seed oil, any oil with fatty acids), antioxidants (for example, vitamin D, vitamin E) and glacial acetic acid. Thus, coconut oil with different concentrations of CA and saline were prepared and placed in vials to allow for polymerization, however, no significant degree of retardation of polymerization was observed. The largest concentration of oil in CA (2:1 oil to CA) polymerized in the catheter with residual saline left behind in the catheter line. Also with the addition of vitamin E to the CA, no significant retardation of polymerization was observed. With a 2:1 concentration of vitamin E to CA, the polymer still cured in the catheter line.

Tests were then conducted using glacial acetic acid to retard polymerization of CA. In these initial tests, acetic acid was mixed with CA and the resulting formulation added to saline in vials in order to see how much the polymerization rate, as well as perhaps other factors, would be delayed. The following table illustrates the polymerization times with different amounts of acetic acid in CA.

TABLE 8
Polymerization times using glacial acetic acid (I)

| Saline (ml) | CA (ml) | Acetic acid (drops) | Polymerization time (mins) |
|---|---|---|---|
| 0.5 | 0.5 | 2 | 7 |
| 0.5 | 0.5 | 1 | 5 |
| 0.5 | 1.0 | 1 | 2 |

Noteworthy, is the nature of the polymerization delay. Acetic acid, more than just slowing the rate of polymerization, seemed to offset or delay the onset of polymerization (this observation was based on a shear qualitative assessment). This finding was noted when after the addition of saline to the CA/acid mixture, no heating would occur for several minutes in the case of 2 drops acetic acid in the mixture. In other words, heating being an indicator of an exothermic reaction due to polymerization, would occur only during the tail end of the trials. An implication of this finding is that an offset or delay would allow the physician to inject glue without having to worry about any immediate increases in viscosity or hardening of the glue solution. On top of effectively retarding the polymerization of CA, this characteristic of offsetting polymerization caused acetic acid to become our prime choice as a retardant.

Having settled on a retardant for cyanoacrylate, we needed to find a procedure and/or methods for injecting this solution, such that:
The glue could be totally delivered without risk of hardening in the catheter during delivery.
The glue solution could harden to a solid mass in the balloon.
CA would not be left in the catheter after its injection into the balloon (for any CA, especially if left uncured could leak out into the parent vessel after detachment).
The polymerization time be long enough so that the physician could inject more saline, if necessary, into the balloon for several minutes after glue injection.
The CA, upon contact with the aneurysmal wall, would result in quick adhesion.
Extreme heating (beyond physiological limits) of the balloon not occur during polymerization.
   Glue Delivery and Inner Balloon Curing Tests

A few device-simulated tests were devised using catheters connected to solid rubber balloons at one end. In one scenario, after injection and removal of saline (to simulate contrast solution), saline was premixed with the CA/acid solution and injected into the catheter. In the second scenario, after injection and removal of saline (to simulate contrast solution), CA/acid solution was injected into the balloon, such that the entire catheter line contained this solution. In the third scenario, after injection and removal of saline, CA/acid solution about equal to the inflated volume of the balloon was injected and chased with saline, such that saline filled the catheter lumen and CA/acid filled the balloon. Note that these tests were developed to investigate the ability to properly deliver glue into the balloon and have it cure inside the balloon and not in the catheter. These tests were not designed to test the device's ability to stick to its periphery.

With the first setup, where 10 drops acid was mixed with 0.75 ml CA and added to 0.75 ml saline then injected, the solution cured during its delivery to the balloon. In the second scenario, the same glue solution reached the balloon and cured in about one minute. However, it did not form into a solid mass. Rather, pockets of solid polymer and others of fluid (mostly acid) were discovered. The glue solution in the catheter did not cure, which is most likely attributable to a deficiency of saline to interact with (the residual saline was most likely pushed up by the glue into the balloon). Thus, detachment of the catheter would most likely result in CA leaking into the parent vessel.

This was confirmed by cutting the catheter line and placing it into a container of saline. A few drops of CA leaked into the saline and polymerized instantly. In the third scheme, all the glue reached the balloon and cured in about one minute into pockets of solid polymer. However, as with the second scenario, a maximum temperature of 47° C. during polymerization of the CA in the balloon was recorded. Since the cured CA in the balloon plugged the end of the catheter connected to the balloon, leakage of CA into the parent vessel after detachment was no longer of a concern (also confirmed by cutting the catheter and placing in a saline bath). These tests demonstrated that the optimal procedure for delivering glue was the third scenario, where glue solution would be chased with saline. Now focus was to be maintained on further delaying the onset of polymerization, as well as devising ways of reducing the maximum temperature of the polymer in the balloon caused by the exothermic reaction. One hypothesis to increase delay was to add more acid.

TABLE 9
Summary of scenarios and their respective outcomes

| Scenario | Procedure | Result |
|---|---|---|
| One | Saline premixed with | Cured in catheter before |
| | CA/acid | reaching balloon |
| Two | Only CA/acid injected | CA/acid did not form solid |
| | | plug in balloon |
| Three | CA/acid chased with saline | CA/acid did not form solid |
| | | plug in balloon |

Thus, greater amounts of acid were added to cyanoacrylate in hopes of greater retarding of polymerization. Using the scheme, as in the second scenario, after injection and removal of saline from a catheter connected to a solid rubber balloon, saline was used to chase the different mixtures of CA/acid solutions such that only saline filled the catheter lumen and residual saline and glue solution filled the balloon. The following table (Table 10) summarises the polymerization times as a result of increased concentrations of glacial acetic acid.

TABLE 10
Polymerization times with glacial acetic acid (II)

| CA (ml) | Acetic acid (drops) | Polymerization time (mins) |
|---|---|---|
| 1.5 | 12 | 1.5 |
| 1.5 | 26 | 5 |
| 1.5 | 28 | 4.5 |

The results show an overall increase in polymerization time with increased concentrations of acetic acid in the glue solution. The small drop in polymerization time between the second and third tests may be attributable to error in time measurements and may illustrate a non-linear relationship, where polymerization time can reach an asymptote with high levels of acid.

Preliminary Glue Adherence Test

To test the binding of the balloon in an aneurysm with such a high acetic glue formulation, 20 drops of acid was mixed with 1.5 ml CA and chased with saline into a porous balloon, which was then placed in a plastic cylinder (to simulate an aneurysm). The balloon was chosen such, so that when inflated, it would completely seal the cylinder (since the balloon was non-compliant, the balloon used was oversized, being 20-30% larger in inflated radius than that of the cylinder). The balloon was bound to the periphery 2 minutes after injection of glue. Qualitative assessments demonstrated that a firm tug on the catheter could not dislodge the balloon out of the tube.

Evaluation of Intermediary Glue Formulation

The benefit of increased acetic acid concentration in the glue formulation was the extended polymerization times, which surpassed the desired time of 4 minutes, as suggested by Dr. Huy Do. However, several problems existed with the current glue solution: great degrees of heating of the balloon during polymerization, polymer curing into pockets within the balloon (for acetic acid was not consumed in the reaction), and because of the persistence of the acid after polymerization, hazards of leaking acid ensued. The pH of our glacial acetic acid (80% acid by volume) was pH 2.3. After one test, where 20 drops acid was added to 1.5 ml CA and injected through a catheter into a balloon, using pH strips, the liquid solution remaining in the balloon post CA polymerization was found to have pH 2.4. Any permeation of this acidic fluid through the pores and leakage into the parent vessel was of great concern. Rupture of the balloon could still occur because it was not forming a solid plug. In which case, the acid and polymer contents would escape into the parent vessel, thus, placing the patient at danger of stroke or other complications attributable to low pH levels in the bloodstream.

Considerations were made of how acetic acid could be used in lower quantities but also have the same effect. Acetic acid, being a hydrophilic solution, could not be mixed with cyanoacrylate into a homogenous solution. Rather, the resulting solution, assuming the mix was agitated prior to injection, was discrete pockets of acid and other of cyanoacrylate. When this non-homogenous mix came in contact with saline, the acetic acid most probably served to decrease the surface area in contact with saline, thus, decreasing the polymerization time of the overall solution. However, it is still not clear whether the acetic acid played any role in actually retarding the polymerization of pockets of CA in contact with saline. Clearly, a method by which the polymerization of CA could be retarded based on reducing the availability of anions to the cyanoacrylate for polymerization was the objective.

Alternative Approach of Retarding Polymerization

One alternative method for retarding the cyanoacrylate, which was developed, was mixing the contrast and chaser saline solutions with acetic acid. Thus, by effectively lowering the pH of the saline, the primary activator for CA polymerization would have more cations than anions available in the solution, thus, perhaps making it more difficult for anions to come in contact with CA. Also, since both acetic acid and saline are hydrophilic solutions, it is possible to form a homogenous solution and ensure that a uniform rate of polymerization occur with all the CA in contact with it.

Catheter-Device Simulated Tests Using Alternative Approach

Twenty drops of acetic acid were mixed with 60 ml saline. The resulting solution was used both for simulating contrast solution and chasing pure cyanoacrylate through the catheter and into a solid rubber balloon. After 30 minutes, polymerization of the CA inside the balloon did not occur nor even initiate. In subsequent tests, the concentration of acetic acid in the saline solution was to be reduced in hopes of achieving polymerization times of 4-5 minutes. In another test, one drop of acid was added and mixed with 30 ml of saline. However, when attempting to use this solution in the catheter-device-simulated setup (catheter with solid rubber balloon), the CA polymerized and occluded the catheter, prior to reaching the balloon. Then, 6 drops acid was added to 60 ml saline. The same procedure was performed in a simulated setup using the resulting solution as the contrast and chaser solutions. The result was that the CA could be fully delivered to the balloon. After 1.5-2 minutes, the CA in the balloon started curing, thus, making it not possible to inject any further chaser saline. It took about 5 minutes for the balloon to cure into a gel material. During this time, no heating of the balloon was observed.

TABLE 11
Summary of tests using catheter-device simulated apparatus with acidic
saline and solid rubber balloon

| | Chaser saline | | Balloon CA | |
|---|---|---|---|---|
| | injection time | | polymerization | |
| Mixture | limit | | time | Comments |
| 20 drops acid | N/A | | N/A | Curing did not initiate |
| with 60 ml | | | | after 30 minutes |
| saline | | | | |
| 1 drop acid | <1 | minute | N/A | CA cured in catheter |
| with 30 ml | | | | prior to reaching |
| saline | | | | balloon |
| 6 drops acid | 1.5-2 | minutes | 5 minutes | Balloon CA cured |
| with 60 ml | | | | into gel. No heating |
| saline | | | | observed |

In a subsequent test, the same formulation was used in a simulated setup with a porous balloon being inserted into a cylinder (to simulate an aneurysm). After about one minute, the balloon adhered strongly to its periphery. After 24 hours, when checked, the balloon had completely hardened forming a solid plug inside the cylinder. Using pH strips, the pH of the saline/acid solution was measured to be about pH 4.5.

Measures were taken to increase the pH of the saline/acid solution and bring it closer to physiological pH. One drop of acid was mixed with 15 ml saline to yield a solution with pH of about 6. The same simulated tests were performed with a porous balloon placed inside a cylinder. After 1 minute, the balloon was bound to its periphery. After 1.5-2 minutes from the injection of the glue, the CA polymerized in the balloon to such a degree than no further chaser saline could be injected into the balloon. After seven minutes, the polymer inside the balloon hardened into a gelly substance. 24 hours post initiation of the test, the polymer inside the balloon was solid. During the first 30 minutes of polymerization, no observable temperature changes of the balloon were observed nor detected by a thermometer. Though we would have preferred longer chaser saline injection times, because of the tradeoffs with pH, this mixture of saline and acid was determined to be our finalist.

Determination of Optimal Mixture of Glue to Weak Activator

Bench tests have demonstrated that saline, as an activator in CA polymerization, is utilized in the reaction. Thus, a set of tests were needed to determine the optimal ratio of saline/acid solution to CA, such that, minimal saline/acid remain in the balloon post polymerization. Tests in vials using the finalist saline/acid solution demonstrated that with a 1:1 mixture of saline/acetic acid solution to CA, minimal solution remained post polymerization (presumably, almost all of it was utilized). Any greater saline/acid solution resulted in excess solution remaining post polymerization, and conversely, any less amount of saline/acid resulted in some CA being left uncured. Thus, the amount of CA injected into the catheter and balloon would have to be based on an approximation of the amount of residual saline in the catheter and balloon, the inflated volume of the balloon, and the total inner lumen volume of the catheter. With knowledge of such parameters, it could be calculated how much CA and chaser solution to inject, such that close to a 1:1 ratio of saline/acid solution to CA could be achieved in the balloon.

Calculation of Injection Volumes of Glue and Weak Activator

If the balloon were solid (such as non-porous) the volume of CA to be injected would need to be about ½ the total inflated volume of the balloon. However, the balloons for this device are porous, thus, some of the CA will leak out into its periphery. Measuring the amount of CA exiting the balloon through the pores would be difficult considering the variations in the geometries of aneurysms. Thus, it shall be assumed that by injecting about ⅔ the total volume of the inflated balloon with CA, that some of it will leave the balloon leaving about ½ the volume of the balloon with CA inside the balloon.

Assuming the volume of residual saline to be negligible in our calculations, the volume of chaser solution needed may be determined, in part, from the inner-lumen volume of the catheter. As an example, assuming a catheter length of 1.5 m and inner radius of 0.5 mm, the volume of the inner-lumen of the catheter would be: Vc=pi*(5E−2 cm)ˆ2*150 cm=1.178 cm³. Thus, the amount of chaser saline required would be Vc plus about ½ the total inflated volume of the balloon. With these amounts of chaser saline and CA, about a 1:1 ratio of saline/acid solution to CA in the balloon can be achieved. Despite what may seem a complex calculation, the device can be marketed with fixed balloon sizes. Each balloon size can come with fixed, premixed amounts of saline/acid solution and CA, in which the respective quantities have been preadjusted as to match the 1:1 ratio.

Alternative Adhesives

(i) RGD

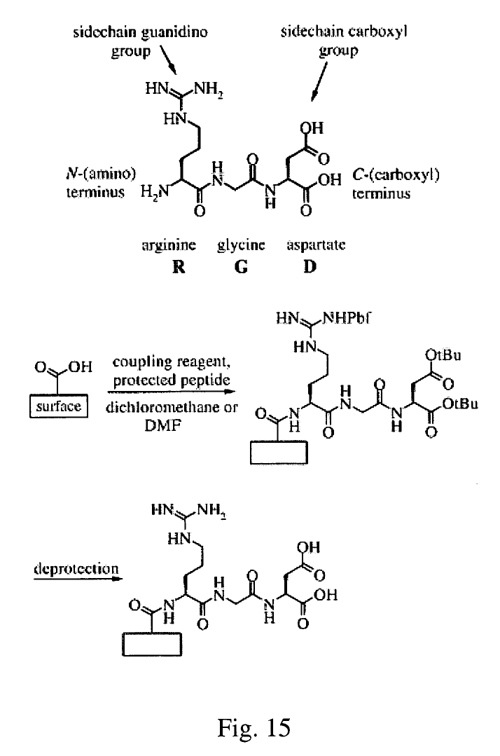

Stable linking of RGD peptides to a synthetic surface is essential to promote strong cell adhesion because focal adhesions formed with the immobilized ligand can withstand the normal contractile forces imposed by the cells. The contractile forces are able to redistribute weakly immobilized ligands and furthermore, internalization of such ligands is thought to induce cell apoptosis. In most cases, RGD peptides are immobilized on polymer surfaces via a stable covalent amide bond. This is usually done by reacting an activated surface carboxylic acid group with the N-terminus of the RGD peptide as shown in FIG. 15. The carboxylic acid groups can be activated using a peptide coupling reagent.

The immobilization of RGD peptides has been simplified by endowing the peptide with a sticky chemical “tail” as in the PEPTITE 2000 by Integra LifeSciences Corporation (Plainsboro N.J.). This provides an easy way to modify different material surface by a simple coating procedure and Table 12 shows a summary of PEPTITE 2000 use.

TABLE 12
Use of PEPTITE 2000 (adapted from Kessler et al. (2003) Biomaterials,
24:4385-4415).

| Amino acid sequence | Polymer | Cell line/tissue |
|---|---|---|
| PepTite 2000 ™ | PTFE | HUVEC |
| | PLGA | Rat |
| | PTFE, PET | Vascular devices (dog, sheep) |

The peptide-polymer surface has been characterized in in-vitro studies to test its effectiveness for cell adhesion and their influence on cell behavior. Cell adhesion to the RGD peptide coated surface is time-dependent. Such adhesion is usually tested 1-4 hours after the seeding of cells onto the surface and increased cell spreading was observed as late as 80 hours later.

(ii) Biocompatible Adhesives

(a) Biological Adhesive Enriched with Platelet Factors;

Contains coagulable human plasma proteins,
Prepared with fibrinogen solutions, which makes it possible to join living tissues while exerting a haemostatic action with the adhesive material,
Adhesive bonding has limited duration because of gradual disappearance of fibrin clot, in vivo, under the action of a proteolytic enzyme called plasmin,
The duration can be reinforced by with alpha-2-antiplasmin or a protease inhibitor such as aprotinin, or alternatively epsilon-aminocaproic acid,
The applications of biological adhesives are numerous, in particular in surgery for avoiding bleeding, for replacing suture threads or for reinforcing sutures.
   (See U.S. Pat. No. 5,589,462.)
   b) Adhesive for Gluing Biological Tissues;
Contains fibrinogen, a substance capable of supplying calcium ions, blood-coagulating factor XIIIa and, as a fibrinogen-splitting substance, a snake-venom enzyme,
Can be used in endoscopic operations, e.g. in the articular field and, in particular, in surgical operations in the vascular field,
Sealing capacity of the adhesive can be considerably increased by the addition of fibronectin to the gluing mixture,
The tensile strength of tissue sealings can be significantly increased if a reduction agent is added to the gluing mixture,
Can lead to inflammation in the adventitia,
Mimics the end stage of plasmatic coagulation, It is known for its strong hemostatic effect,
It is effective in controlling bleeding,
When applied around aneurysms, the glue is often absorbed by the circulation in the vessel.

(See U.S. Pat. No. 6,613,324; Herrera et al. (1999) Neurol. Med. Chir. (Tokyo) 39: 134-139; discussion 139-140; Lee et al. (1991) Yonsel Medical Journal, 32(1).)

TABLE 13
Examples of Combining Biological Adhesives

| | | | | | | Maximum |
|---|---|---|---|---|---|---|
| | | | | | | tensile |
| | Fbg | FN | FXIIIa | Ba | Thr | strength |
| Experiment | (mg/ml) | (mg/ml) | (ml) | (ml) | (ml) | (N/cm²) |
| 1 | 20 | 2 | 1.6 | 5.4 | 0 | 8.8 |
| 2 | 20 | 0 | 1.6 | 5.4 | 0 | <20 |
| 3 | 20 | 2 | 0 | 5.4 | 0 | <20 |
| 4 | 20 | 2 | 1.6 | 0 | 4 | 3.6 |
| Control | 0 | 0 | 0 | 0 | 0 | <20 |

Notes:
Fbg = fibrinogen,
FN = fibronectin,
Ba = batroxobin,
Thr = thrombin

TABLE 14
Examples of Combining Biological Adhesives and DTT

| | | | | | | Maximum |
|---|---|---|---|---|---|---|
| | | | | | | tensile |
| | Fbg | DTT | FN | FXIIIa | Ba | strength |
| Experiment | (mg/ml) | (mM) | (mg/ml) | (ml) | (ml) | (N/cm²) |
| 1 | 20 | 0 | 2 | 1.6 | 22 | >9.0 |
| 2 | 20 | 0 | 2 | 1.6 | 22 | >9.0 |
| 3 | 20 | 0.5 | 2 | 1.6 | 22 | >9.0 |
| 4 | 20 | 0.5 | 2 | 1.6 | 22 | >9.0 |
| 5 | 10 | 0 | 1 | 1.6 | 22 | 5.0 |
| 6 | 10 | 0 | 1 | 1.6 | 22 | 5.0 |
| 7 | 10 | 0.5 | 1 | 1.6 | 22 | >9.0 |
| 8 | 10 | 0.5 | 1 | 1.6 | 22 | >9.0 |

Notes:
Fbg = fibrinogen,
DTT = dithiothreitol,
FN = fibronectin,
Ba = batroxobin

(c) Ultrasonographic-Guided Glue;
Used in the treatment of femoral pseudoaneurysms,
The aneurysm neck is compressed during glue injection to prevent distal embolization,
Injection is performed in conjunction with ultrasonographic guidance,
Procedure time varies between 5 and 20 minutes,
All cited cases were carried through successfully.
   (See Aytekin et al. (2003) Tani Girisim Radyol. 9: 257-259.)
   (d) Adhesive Composition Resistant to Biological Fluids;
Comprising a homogeneous mixture of one or more polyisobutylenes or blends of one or more polyisobutylenes and butyl rubber, one or more styrene radial or block type copolymers, mineral oil, one or more water soluble hydrocolloid gums, and a tackifier,
Medical grade pressure sensitive adhesive compositions,
Adapted for use in the fields of incontinence, ostomy care and wound and burn dressings,
Compositions of this invention are resistant to erosion by moisture and biological fluids,
Can be employed in multilayered occlusive dressings.
   (See U.S. Pat. No. 4,551,490.)
   (e) Biological Adhesive Composition Promoting Adhesion Between Tissue Surfaces;
Utilizes tissue transglutaminase in a pharmaceutically acceptable aqueous carrier adhesive composition may be employed in grafting (repairing) nerves and blood vessels, patching vascular grafts, and microvascular blood vessel anastomosis, May include the pretreatment of tissue surfaces with digestive enzymes may be used to enhance adhesion, Key substance is tissue transglutaminase, an enzyme that catalyses a chemical reaction by which proteins become crosslinked to form network-like polymers.
   (See U.S. Pat. No. 5,549,904.)
   (f) Adhesive for Biological Tissue Including a Glue Agent and Cross-Linking Agent;
Provides good adhesion strength,
There are possibilities of infection with viruses,
Agent “A” contains a 45 wt % aqueous solution of recombinant human serum albumin. Agent “B” contains an aqueous solution containing recombinant human serum albumin in the amount of 25 wt %. Agent “C” contains an aqueous solution containing recombinant human serum albumin in the amount of 30 wt %.

(See U.S. Pat. No. 6,329,337.)

TABLE 15
Examples of Combining Biological Adhesives and Cross-linking Agents

| | | Conc. of cross- | |
|---|---|---|---|
| | | linking agent (wt | Tensile strength |
| Test No | Glue agent | %) | (g/cm²) |
| 1 | A | 2.5 | 600 |
| 2 | A | 5.0 | 750 |
| 3 | A | 10.0 | 1,200 |
| 4 | B | 2.5 | 800 |
| 5 | B | 5.0 | 1,000 |
| 6 | C | 2.5 | 650 |
| 7 | C | 5.0 | 800 |
| 8 | Conventional fibrin | | 200 |
| | glue (Bolheal) | | |

(g) Gelatin-Resorcin-Formaldehyde (GRF) Glue;
Currently used to reinforce dissected aortic wall, or alternatively to the anastomotic site for hemostasis. Also has been used in thoracic aortic operations,
Often times glue fails to harden,
With agitation of formulation, optimal hardening may be achieved.
   (See Nishimori et al. (2000) Ann. Thorac. Surg. 69: 1295-1302.)
   Calculation of Radial Force Exerted by Adjacent Blood Flow on Balloon in the Carotid Artery

Assume maximum blood pressure (during peak systole in hypertensive individuals) in the carotid artery is P=120 mm Hg.

Converting units:
P=(120 mm Hg)(101325 N/m²)/(760 mm Hg)=15998.7 N/m²

Stress exerted by blood flow on balloon in radial direction (component of stress tensor with plane normal to radial axis and stress component in radial direction):
Trr=P=15998.7 N/m²

This assumes that stress is constant over the front face of the cylindrical balloon. In reality, this is an approximation because the balloon surface is flat while the vessel wall is not, hence, in vivo a small stress gradient will exist on the balloon surface.

Surface area of balloon in contact with blood (consider 10 mm diameter cylindrical balloon):
A=pR²=p(10 mm/2)²=78.54 mm²

Force exerted on balloon by adjacent blood flow:
Fb=Trr*A=15998.7 N/m²*78.54 mm²*(1 m/1000 mm)² [symbol]
Fb≈1.26 N
Detachment

Failure of proper electrolytic detachment can be considered a device failure mode since surgical intervention then may be required to remove the catheter. Because it cannot be ensured that only chaser saline remain the catheter lumen at the segment of the steel coupling during detachment, tests were needed to investigate the functionality of electrolysis under different possible scenarios.

Three possible fluids, any of which, that could end up in the steel coupling segment during detachment were considered: saline, air and cyanoacrylate. Three 1 mm diameter catheters with steel couples (but deficient of balloons) were soldered to 0.2 mm diameter copper wires and were placed in a saline bath and tested with each of the different fluids in their respective lumens. Each copper wire was connected to a DC power source and a 90 mA current applied through the wires. In each test, detachment occurred within 2-3 minutes of initiation. Thus, it was determined that electrolytic detachment would occur despite what fluid or combination of fluids (and polymer) remained in the steel coupling segment of the catheter.

It will be readily appreciated that various adaptations and modifications of the described embodiments can be configured without departing from the scope and spirit of the invention and the above description is intended to be illustrative, and not restrictive, and it is understood that the applicant claims the full scope of any claims and all equivalents.

1. A device for occluding an aneurysm comprising: a detachable, semi-compliant, radially-expanding balloon mounted on a catheter, wherein the catheter comprises a catheter body defining at least one interior lumen, wherein the balloon is in fluid communication with at least one lumen defined within the catheter, wherein the balloon comprises a plurality of micropores, wherein the micropores in the balloon allow expression of an adhesive fluid at a defined pressure from the inside to the outside of the balloon.

2. The device of claim 2 wherein the micropores are disposed unevenly upon the surface of the balloon.

3. The device of claim 2 wherein the majority of the micropores are disposed on the upper hemisphere of the balloon.

4. The device of claim 2 wherein the micropores are disposed over an area of not more than 50% or the surface of the balloon.

5. The device of claim 2 wherein the micropores are disposed over an area of not more than 30% or the surface of the balloon.

6. The device of claim 2 wherein the micropores are disposed over an area of not more than 10% or the surface of the balloon.

7. The device of claim 2 wherein the total combined surface area of the micropores is not more than 1% of the total surface area of the balloon.

8. The device of claim 2 wherein the total combined surface area of the micropores is not more than 2% of the total surface area of the balloon.

9. The device of claim 2 wherein the total combined surface area of the micropores is not more than 5% of the total surface area of the balloon.

10. The device of claim 2 wherein the fluid is a bio-adhesive fluid that solidifies under physiological conditions.

11. The device of claim 10 where the fluid is a polymerizing material.

12. The device of claim 11 wherein the fluid is a cyanoacrylate material.

13. The device of claim 2 wherein the shape of the balloon is approximately torroidal.

14. The device of claim 2 wherein the shape of the balloon is disc-shaped wherein the diameter of the disc is greater than the thickness of the disc.

15. The device of claim 14 wherein the disc-shaped balloon possesses a concave lower surface.

16. The device of claim 2 wherein the catheter comprises a major lumen and a minor lumen wherein the major lumen is adapted for delivery of the bio-adhesive fluid and wherein the minor lumen is adapted for containment of an electrically conductive wire.

17. The device of claim 16 further comprising, at or near the attachment point of the balloon and the catheter body, a steel coupling detachably joining the balloon and the catheter body.

18. The device of claim 2 wherein expression of the bio-adhesive fluid from the micropores requires a minimum interior pressure of 30 mm Hg.

19. The device of claim 18 wherein expression of the bio-adhesive fluid from the micropores requires a minimum interior pressure of to 60 mm Hg.

20. The device of claim 18 wherein expression of the bio-adhesive fluid from the micropores requires a minimum interior pressure of to 80 mm Hg.

21. The device of claim 18 wherein expression of the bio-adhesive fluid from the micropores requires a minimum interior pressure of to 100 mm Hg.

22. The device of claim 18 wherein expression of the bio-adhesive fluid from the micropores requires a minimum interior pressure of to 120 mm Hg.

23. The device of claim 18 wherein expression of the bio-adhesive fluid from the micropores requires a minimum interior pressure of to 160 mm Hg.

24. The device of claim 2 wherein the diameter of the micropores is between 1 μm and 10 μm.

25. The device of claim 1 wherein the balloon comprises a non-compliant material.

26. A method of using a device for occluding an aneurysm in an individual, the method comprising the steps of: i) providing an individual at risk for having an aneurysm; ii) providing the device of claim 1; iii) inserting a guidewire through a blood vessel of the individual into the aneurysmal space; iv) using the guidewire as a rail inserting the device through the blood vessel; v) advancing the device until the balloon is positioned in the aneurismal space; vi) injecting a radio-opaque composition into at least one lumen of the device; vii) visualizing the radio-opaque composition in the aneurysmal space; viii) withdrawing the radio-opaque composition from the device; ix) injecting a adhesive fluid into the device at a pressure suitable for inflating the balloon and fixing the balloon against the interior wall of the aneurysm; x) placing an electrode on the individual, the electrode being in electrical communication with the ground attachment of a voltage source; xi) applying a potential difference to the electrically conducting wire thereby causing electrolysis of the steel couple and releasing the steel couple from the non-steel couple; thereby treating the aneurysm.
